(19) United States
(12) Patent Application Publication
(10) Pub. No.: US 2007/0299021 A1
(43) Pub. Date: Dec. 27, 2007

(54) Modified Tailed Oligonucleotides

(76) Inventors: Matthew Graeme Dunckley, London (GB); Ian Charles Eperon, Leicester (GB); Francesco Muntoni, London (GB)

Correspondence Address: JAECKLE FLEISCHMANN & MUGEL, LLP, 190 Linden Oaks, ROCHESTER, NY, 14625-2812, US

(21) Appl. No.: 10/524,724

(22) PCT Filed: Aug. 18, 2003
(86) PCT No.: PCT/GB03/03612

§ 371 Date: Oct. 18, 2006

(30) Foreign Application Priority Data
Aug. 16, 2002 (GB) 0219143.5

(51) Int. Cl. A61K 48/00; A61K 31/7105; A61P 43/00; C07H 21/02; C12N 15/10; C12N 15/11; C12N 15/85; C12N 5/10

(52) U.S. Cl. 514/44; 435/320.1; 435/325; 435/375; 536/23.1; 536/24.1; 536/25.3

(57) ABSTRACT

A nucleic acid molecule comprising first and second domains, said first domain being capable of forming a first specific binding pair with a target sequence of a target RNA species, said second domain consisting of a sequence which forms a second specific binding pair with at least one RNA processing or translation factor, said target sequence being sufficiently close on said target RNA species to an RNA processing or translation site for processing or translation at said site to be enhanced by the action of the factor bound to the second domain.

The present invention relates to modified nucleic acid molecules that are used to provide positively acting RNA processing signals in trans.

Antisense methods are widely used to inhibit gene expression in eukaryotic cells. From the therapeutic point of view, one of the most promising developments has been the use of modified and more stable oligonucleotides, for example 2′-O-methyl derivatives of RNA, which can be taken up by cells and will anneal to a specific target mRNA to block its expression. In principle, any target gene can be down-regulated by such reagents. A variation of the method has been used to prevent the incorporation of a specific block of RNA into the mature mRNA by preventing splicing of particular exons from the precursor (pre)-mRNA molecule. This may have therapeutic uses in some diseases, such as muscular dystrophy. For example, Dunckley et al have shown that a severe dystrophy caused by a mutation that introduced a translational stop coden could be alleviated in principle by the use of antisense oligonucleotides that blocked the splicing of that exon (Dunckley, M. G., Manoharan, M., Villiet, P., Eperon, I. C., and Dickson, G. (1998) Hum. Mol. Genet. 7(7), 1083-1090, PMID: 9618164). Lu et al (Nature Medicine (2003) vol. 9(8): 1009-1014) produced functional amounts of dystrophin by skipping the mutated exon in the mdx dystrophic mouse, demonstrating that this principle works in vivo in mammals.

Practically all of the existing methods of modifying the expression of endogenous genes result in a reduction of expression or reduction in the incorporation of particular (deleterious) exons. Short of introducing a correct gene, no general methods are available for enhancing expression or correcting the effects of splicing-related mutations on the basis of knowing only the sequences of the wild-type and (if any) related genes.

Alternative pre-mRNA splicing is a fundamental mechanism for regulating the expression of a multitude of eukaryotic genes. The basic splicing signals, which include the 5′ splice site, branch site, and polypyrimidine tract-AG, are initially recognized by the U1 small nuclear ribonucleoprotein (snRNP), U2 snRNP, U2 snRNP auxiliary factor (U2AF), respectively, and a number of other proteins. These basic splicing signals tend to be degenerate in higher eukaryotes and cannot alone confer the specificity required to achieve accurate splice site selection. Various types of exonic and intronic elements that can modulate the use of nearby splice sites have now been identified. Among the best known examples of such elements are the exonic splicing enhancers—sequences naturally present in pre-mRNA that stimulate the splicing of pre-mRNA transcripts to form mature mRNAs (Cartegni, L. et al (2002) Nat. Rev. Genet. 3(4), 285-298, PMID: 11967553; Cáceres, J. F. and Kornblihtt, A. R. (2002) Trends Genet. 18(4), 186-193, PMID: 11932019). The definition of “enhancer” is functional, and includes sequences within exons that are not located at the splice sites and are not universally obligatory but do stimulate splicing at least in the gene in which they were identified. Enhancers are commonly thought of as elements in alternatively spliced exons that compensate in part for weak canonical splicing signals. However, it has been shown recently that even constitutive exons can contain several enhancer sequences. The majority of enhancer sequences identified are rich in purines, although recent selection strategies have shown that more diverse classes of sequence are also functional. In a number of cases, it has been shown that these sequences are recognised directly by specific SR (for serine and arginine-rich) proteins. These RNA-binding proteins play a critical role in initiating complex assembly on pre-mRNA, and are essential fox constitutive splicing and also affect alternative splicing both in vivo and in vitro. It is very likely that other proteins, such as Tra2α or β or hnRNP G also play a role in enhancer sequence recognition and/or processing.

Enhancer sequences have also been identified in introns, however general principles concerning their sequence or mode of action have yet to emerge.

In all known cases, enhancer sequences act in cis, i.e. they are part of the pre-mRNA substrate. Enhancers can act in cis within a partial substrate, where a substrate lacking a 3′ exon has undergone the first step of splicing and then a second RNA containing the 3′ portion and an enhancer is added. However, there have been no reports of enhancers acting positively in trans, and indeed, enhancers are often added in trans as competitors to titrate out enhancer binding factors.

Pre-mRNA molecules may also contain cryptic or mutant splice sites, especially 5′ splice sites. The 5′ splice site is defined by a poorly conserved short sequence around a highly conserved GU (guanine-uracil) dinucleotide. In most cases, there are many similar sequences in the adjacent intron and exon, but the correct site is chosen as a result of a combination of influences: the extent to which the sequences fit the consensus, the positions of exon elements and other splice sites, and the concentration of the various factors that affect 5′ splice sites. Numerous genetic diseases result from mutations at the 5′ splice site, the consequences of which are either skipping of the exon or the use of some of the other candidate sites (cryptic splice sites). Enhancer defects are difficult to assign and have only recently entered the broader consciousness as possible explanations for the effects of mutations. Well-known examples of genetic diseases that arise from mutations affecting splicing include thalassaemias (e.g. OMIM #141900 for haemoglobin-beta locus), muscular dystrophies (e.g. OMIM #310200), collagen defects (van Leusden, M. R. et al (2001) Lab Invest. 81(6), 887-894, PMID: 11406649), and proximal spinal muscular atrophy (SMA) (Monani, U. R., et al (1999) Hum. Mol. Genet. 8, 1177-1183, PMID: 10369862; Lorson, C. L., et al (1999) Proc. Natl. Acad. Sci. USA 96, 6307-6311, PMID: 1 0339583).

SMA is an autosomal recessive disorder characterized by muscular weakness and atrophy due to the degeneration of spinal cord motor neurons resulting from mutations of the Survival Motor Neuron (SMN) gene. The SMN gene consists of eight exons, the first seven of which encode a 294 amino acid protein with a molecular weight of 32 kDa. The SMN protein is ubiquitously expressed and localised in the cytoplasm and nucleus where it is involved in the process of pre-mRNA splicing. In particular it has a role in the recycling of snPNPs in the nucleus and probably also in spliceosomal snRNP assembly in the cytoplasm. The SMN gene exists in two copies, a telomeric (SMN1) And a centromeric copy (SMN2). Mutations in SMN1 cause SMA, while the copy number of the residual SMN 2 genes is believed to modify the severity of the phenotype. In support of this hypothesis, it has been shown that an increased copy number is associated with a milder disease course. Deletions of beth SMN1 and SMN2 have never been observed in humans and a knockout of the single SMN gene in the mouse results in a non-viable embryo. The two genes are 99% identical and differ only by 8 nucleotides, only 2 of which are contained in exons and neither of which alters the coding sequence. The SMN1 and SMN2 genes undergo alternative splicing involving exon 7 and to a lesser extent exon 5, resulting in the SMN1 gene producing primarily full-length SMN transcript whereas the predominant transcript derived from SMN2 lacks exon 7. One of these nucleotide changes is C6T—a T for C substitution at position +6 in exon 7 of SMN2. This nucleotide is essential for the retention of exon 7 in the mature transcript of the SMN1 gene. This is accomplished by the presence of a high affinity binding site in the SMN1 gene for the SR protein SF2/ASF which generally promotes the inclusion of exons to which it binds (Hastings, M. L. and Krainer, A. R. (2001) Curr. Op in. Cell Biol. 13(3), 302-309, PMID: 11343900). One explanation for this observation is that the C6T change found in SMN2 abolishes the ability of this region to bind SF2/ASF, thereby reducing the recognition of exon 7 by the spliceosome, resulting in exon 7 deleted SMN2 transcripts (Cartegni, L. and Krainer, A. R. (2002) Nature Genetics 4, 377-384, PMID: 11925564). Alternatively, the C6T change may introduce a silencer into SMN2 exon 7 which inhibits splicing (Kashima T & Manley J L, Nature Genetics Jun. 29, 2003 [Epub ahead of print]).

The retention of intact copies of SMN2 in all SMA patients has led various investigators to devise different strategies for altering the splicing pattern of the SMN2 gene to that of the SMN1 gene, as this might have therapeutic implications for SMA patients. This has been attempted by using pharmacological agents such as sodium butyrate and aclarubicin (Chang, J. G. et al (2001) Proc. Natl. Acad. Sci. USA 17, 9808-9813, PMID: 11504946; Andreassi, C. et al (2001) Hum. Mol. Genet. 24, 2841-2849, PMID: 11-734549) or antisense strategies, with oligonucleotides targeted against exon 8 splice sites, thereby blocking the sites and inducing exon 7 inclusion to a greater extent (Lim, S. R. and Hertel, K. J. (2001) J. Biol. Chem. 276(48), 45476-45483). However, only a very moderate increase in exon 7 inclusion was achieved by this antisense approach and the drugs involved have potential toxicity problems. Furthermore, the use of antisense oligonucleotides to block an adjacent exon is applicable only in rare cases where this is the 3′ terminal exon—if it were an internal exon, the antisense oligonucleotide might lead to skipping of the blocked exon.

The present invention aims to overcome at least one of the prior art disadvantages and contributes significantly to the field, for example by providing a novel product and method for overcoming genetic or induced mutations in RNA molecules that prevent the recruitment of endogenous processing factors to the RNA molecules. An oligonucleotide molecule that comprises an RNA binding domain and an RNA processing factor binding domain is introduced into cells carrying the defective RNA species. The oligonucleotide molecule anneals by means of the RNA binding domain to specific RNA sequences at or near the defective site, and then by means of the RNA processing factor binding domain recruits endogenous RNA processing factors which interact with said RNA species, thereby overcoming the effect of the mutation. This method is universally applicable and requires no further characterisation beyond knowledge of the mutation.

In some cases, the splicing pattern of a ‘normal’ or unmutated gene is altered, leading to a disease phenotype. The novel product and method can be used to correct inappropriate splicing of a gene, for example one which is associated with a disease condition such as inflammation, or indeed to stimulate exon incorporation in disease gene for therapeutic benefit.

Thus, according to a first aspect of the present invention, there is provided a nucleic acid molecule comprising first and second domains, said first domain being capable of forming a first specific binding pair with a target sequence of a target RNA species, said second domain consisting of a sequence which forms a second specific binding pair with at least one RNA processing or translation factor.

The nucleic acid molecule may be considered to be a gene-specific trans-acting enhancer of RNA processing or translation.

Thus the first domain of the nucleic acid molecule is an RNA binding domain and the second domain is an RNA factor binding domain.

The first domain of the nucleic acid molecule is designed to bind to the target sequence on the target RNA species sufficiently close to am RNA processing or translation site in the target RNA species for processing or translation at the site to be enhanced by the action of the second domain, ie by the binding of the second domain to the RNA processing or translation factor, thus recruiting the factor to the RNA processing or translation site.

The skilled person would readily appreciate that there are practical constraints on the size of the first domain of the nucleic acid molecule. If it is too short the binding to the target sequence would be unstable; if it is too long there is an increased possibility that part of the first domain will anneal to other targets. It is preferred if the full length of the first domain anneals to the target region of the target RNA species to maximize specificity of binding. Thus, typically, the first domain of the nucleic acid molecule is from 8 to 50 nucleotides in length. The first domain can be 8, or 9, or 10, or 11, or 12, or 13, or 14, or 15, or 16, or 17, or 18, or 19, or 20 to 25, or 26 to 30, or 31 to 40, or 41 to 50 nucleotides in length. Preferably, it is between 10 to 25 nucleotides in length.

Typically, the first domain of the nucleic acid molecule binds to the target sequence on the target RNA species by complementary base pairing. Preferably, the first domain has at least 90% sequence identity with the target sequence, more preferably at least 95% or at least 99% sequence identity. It is most preferred if the first domain has 100% sequence identity with the target sequence. When the first domain is between 10 to 25 nucleotides in length, it requires a higher level of sequence identity with the target sequence, and preferably having only a single mismatch or none at all. However, with a longer first domain, such as 50 nucleotides or more, a lower level of sequence identity with the target sequence may be acceptable.

It is preferred if the target sequence occurs only once in the target RNA species. It is also preferred if the target sequence only occurs once in the genome of the organism from which the target RNA is expressed.

Typically, the nucleic acid molecule is arranged such that upon formation of a first specific binding pair with said target sequence, the at least one RNA processing or translation factor interacts with the RNA target species at the RNA processing or translation site to effect RNA processing or translation at the RNA processing or translation site.

It is appreciated that the second domain of the nucleic acid molecule can form a second specific binding pair with the RNA processing or translation factor before, after or substantially simultaneously with the formation of the first specific binding pair.

The second domain of the nucleic acid molecule should not be complementary to the RNA target species, so that it is available for the binding of RNA processing factors.

Typically, the second domain of the nucleic acid molecule is typically from 5 to 50 nucleotides in length, and may be longer Thus the second domain can be 5, or 6, or 7, or 8, or 9, or 10, or 11, or 12, or 13, or 14, or 15, or 16, or 17, or 18, or 19, or 20, to 25, or 26 to 30, or 31 to 40, or 41 to 50 or more nucleotides in length. The minimum binding site for an RNA processing or translation factor is three nucleotides although to allow accessibility to the factors, a minimum size for this domain would be around 5 nucleotides. However, the optimal size is typically higher. The length of the second domain may be increased by including tandem repeats or arrays of recognition motifs for the RNA processing or translation factor, to minimise spurious binding.

Thus the entire nucleic acid molecule is typically from 13 to 100 nucleotides or more in length. Preferably, the entire nucleic acid molecule is from 15 to 50 nucleotides in length, and can be, for example, 15 or 16, or 17, or 18, or 19, or 20, or 21, or 22, or 23, or 24, or 25, or 26, or 27, or 28, or 29, or 30, or 31 to 40, or 41 to 50 or more nucleotides in length.

Thus the invention includes a nucleic acid molecule comprising first and second domains, said first domain being capable of forming a first specific binding pair with a target sequence of a target RNA species, said second domain consisting of a sequence which forms a second specific binding pair with at least one RNA processing or translation factor, said target sequence being sufficiently close on said target RNA species to an RNA processing or translation site for processing or translation at said site to be enhanced by the action of said second domain, and said nucleic acid molecule being arranged such that upon formation of a first specific binding pair with said target sequence, said at least one RNA processing or translation factor interacts with said RNA target species to form a second specific binding pair at said RNA processing or translation site to effect RNA processing or translation at said RNA processing or translation site.

It is appreciated that the RNA processing or translation factor does not necessarily interact directly with the RNA target species at the RNA processing or translation site. In most cases, the factor that is recruited to the RNA processing or translation site ‘complexes’ with other proteins or ribonucleoproteins on the target RNA species. For example, correct splicing requires the coordinated action of five small nuclear RNAs and more than 60 polypeptides (see, Cartegni et al (2002) Nature Reviews Genetics 3(4): 285-298, and the references cited within).

Thus the invention includes a nucleic acid molecule comprising first and second domains, said first domain being capable of forming a first specific binding pair with a target sequence of a target RNA species, said second domain consisting of a sequence that forms a second specific binding pair with at least one RNA processing or translation factor, said target sequence being sufficiently close on said target RNA species to an RNA processing or translation site for processing or translation at said site to be enhanced by the action of the factor bound to the second domain.

Regarding the proximity of the target sequence to the RNA processing or translation site on the target RNA species, as used herein, the terms “sufficiently close”, “near to” and “close to” may mean between 0 and 1,000 nucleotides, more preferably between 0 and 500 nucleotides, still more preferably between 0 and 200 nucleotides, and yet more preferably between 0 and 100 nucleotides. For example, the target sequence may be 0, 1, 2, 3, 4, or 5, 6, 7, 8, 9, or 10, 15, 20, 25, 30, 35, 40, 45, 50, 55, 60, 65, 70, 75, 80, 85, 90, 95, or 100 nucleotides from the RNA processing or translation site. However, RNA is known to form a range of secondary structures which may bring the target sequence on the target RNA species sufficiently close to the RNA processing or translation site for processing or translation at the site to be enhanced by the action of the factor bound to the second domain, even if the target sequence and the RNA processing or translation site are separated by many kilobases apart on the target RNA species.

A “Member of a Specific Binding Pair” is one of two different molecules, having an area on the surface or in a cavity which specifically binds to the other molecule with a particular spatial and polar organization. The members of the specific binding pair are referred to as ligand and receptor (antiligand), sbp member and sbp partner, sbp members or the like. The members of the first specific binding pair can be nucleic acid duplexes, such as RNA-RNA duplexes. The members of the second specific binding pair can be nucleic acid duplexes, nucleic acid-protein, RNA-RNA, RNA-protein (including RNA-ribonucleoprotein), and the like. It is appreciated that to form a specific binding pair, the two molecules associate with sufficient specificity and affinity for the interaction to be useful.

Preferably, the second domain of the nucleic acid molecule has a sequence binding motif that is recognised by the RNA processing or translation factor allowing the formation of the second specific binding pair with the factor.

RNA processing factors may be any RNA or protein that stimulates splicing activity or translation when recruited to the RNA target species at the RNA processing or translation site. The RNA processing factors may- comprise any one of the group of: RNA molecules, RNA structural molecules, RNA stability molecules, splicing factors, polyadenylation factors, transcription factors, and translation factors. These factors may include cellular proteins, nucleic acids, ribonucleoprotein complexes, and combinations thereof.

RNA splicing factors may comprise any one of the group of proteins that influence the site or efficiency of splicing, such as SR proteins, SR-related proteins (Graveley, B. R. (2000) RNA 6(9): p 1197-1211, PMID: 10999598), or hnRNP proteins (Krecic, A. M. and Swanson, M. S. (1999) Curr. Opin. Cell Bio. 11(3): p 363-371, PMID: 10395553). The RNA sequence binding motifs associated with these proteins are well characterised and are known to a person skilled in the art. Further splicing enhancer sequences known in the prior art (supra) may also be utilised.

RNA motifs that are recognised by human SR proteins are listed in Table 1 in Cartegni, et al, (2002) Nat. Rev. Genet. 3, 285-298, incorporated herein by reference.

Apart from the SR-dependent enhancers, numerous sequences in introns or exons have been shown to affect splice site selection or exon incorporation. In some cases, these affect the processing of specific target gene transcripts in precise ways (reviewed by Smith & Valcarcel, Trends Biochem Sci 25, 381-388 (2000)). However, many of them are bound by hnRNP proteins, which are known to bind nascent transcripts, to be at least reasonably abundant and, often, to be expressed ubiquitously (Krecic & Swanson, Curr Opin Cell Biol 11, 363-371 (1999)), leading to the supposition that they will in fact recognise sequences in numerous transcripts and influence splicing rather widely. Other sequence elements defined recently include (A+C)-rich enhancers, found recently to be recognised by the protein YB-1 52 (Stickeler et el., Embo J 20, 3821-3830. (2001); intronic GGG triplets, recognised by U1 snRNA (McCullough & Berget, Mol Cell Biol 20, 9225-9235. (2000)); GGGGCUG sequences that are recognised by mBBP (Carlo et al, Mol Cell Biol 20, 3988-3995. (2000)); and purine-rich sequences recognised by T-STAR, a possible mediator of signalling responses identified by this laboratory (Venables et al. Hum Mol Genet 8, 959-969 (1999)) and then shown to affect splicing (Stoss et al. J Biol Chem 276, 8665-8673. (2001)). RNA splicing factors also include STAR proteins, CELF proteins, peliotropic proteins such as YB1, nuclear scaffold proteins and helicases.

A useful motif for the second domain of the nucleic acid molecule is CAGGUAAGU which is the binding site for the U1 snRP.

It is appreciated that the second domain may contain sequence binding motifs that are known to enhance RNA processing or translation, such as splicing, even if the RNA processing or translation factor which recognises these motifs has not yet been identified. For example, Fairbrother et al, (2002, Science 297 (5583): 1007-1013) identified ten exonic splicing enhancer sequence motifs in human genes, each of which may be suitable for inclusion in the second domain.

It is appreciated that the motifs for inclusion in the second domain are consensus motifs and include functional variants clustered around an optimum sequence.

In an embodiment, the invention includes a nucleic acid molecule comprising first and second domains, said first domain being capable of forming a first specific binding pair with a target sequence of a target RNA species, said second domain comprising the sequence AGGAGGACGGAGGACGGAGGACA (SEQ ID NO: 16).

In other embodiments, the second domain may contain other GGA repeat motifs which may act as a recognition site for the SF2/ASF factor.

The nucleic acid molecule may contain multiple functional domains, for example, it may contain binding sites for at least one RNA processing or translation factor such as an SR or SR-related protein (see, for example, Hertel & Maniatis (1998), “The function of multisite splicing enhancers” Molecular Cell 1(3): 449-55).

In an embodiment, the nucleic acid molecule may also contain at least one domain to facilitate coupling of the oligonucleotide to additional compounds to enhance uptake into cells and nuclei, eg a penetratin binding domain (Derossi, D. et al (1998) Trends Cell Biol. 8(2): p 84-87, PMID: 9695814; Derossi, D. et al (1994), J. Biol. Chem. 269(14): p 10444-10450, PMID: 8144628).

The nucleic acid molecule may also contain a 3′ hairpin to improve stability or a modified 5′ end to avoid degradation.

The nucleic acid molecule may be isolated and/or purified.

The nucleic acid molecule according to the present invention may be prepared by any convenient method involving coupling together successive nucleotides, and/or ligating oligo- and/or poly-nucleotides, including in vitro processes, as well as by recombinant DNA technology (Sambrook, J., Frisch, E. F. and Maniatis, T. (1989) Molecular Cloning: A Laboratory Manual, Cold Spring Harbor Laboratory, Cold Spring Harbor, N.Y.).

The nucleic acid :molecule may be introduced into target cells using well-known transformation and transfection techniques and reagents eg Lipofect Amine (Life Technologies), or GeneJammer (Stratagene), used according to the manufacturers instructions.

By “nucleic acid molecule” we include the meaning of a consecutive series of bases, usually found naturally in RNA, that can form specific base-pairs with a complementary sequence. The bases may be connected by a ribose-phosphodiester backbone or other linking units.

Typically and preferably, the nucleic acid molecule is an RNA molecule, ie it is an oligoribonucleotide. Preferably, the nucleic acid molecule is not DNA as this would trigger ribonuclease H degradation of the target RNA species. The nucleic acid molecule may include phosphoramidate linkages which improve stability, the free energy of annealing and resistance to degradation (Faria et al, 2001, Nature Biotechnol. 19(1): 40-44); or locked nucleic acids (LNA, Kurreck et al, 2002, Nucleic Acids Res. 30(9): 1911-8), or peptide nucleic acids (PNA).

The nucleic acid molecule can comprise at least one modified nucleotide. For example, at least one nucleotide may be chemically modified to enhance stability. Examples of modified nucleotides include those listed in WIPO standard ST25. At least one nucleotide may for example be a 2′-O-methyl derivative of RNA and/or a phosphorothioate or morpholino modification. Such modified nucleotides are more stable and less susceptible to attack by endogenous RNAses and other cellular degradation processes.

Oligonucleotides are subject to being degraded or inactivated by cellular endogenous nucleases. To counter this problem, it is possible to use modified oligonucleotides, eg having altered internucleotide linkages, in which the naturally occurring phosphodiester linkages have been replaced with another linkage. For example, Agrawal et al (1988) Proc. Natl. Acad. Sci. USA 85, 7079-7083 showed increased inhibition in tissue culture of HIV-1 using oligonucleotide phosphoramidates and phosphorothioates. Sarin et al (1988) Proc. Natl. Acad. Sci. USA 85, 7448-7451 demonstrated increased inhibition of HIV-1 using oligonucleotide methylphosphonates. Agrawal et al (1989) Proc. Natl. Acad. Sci. USA 86, 7790-7794 slowed inhibition of HIV-1 replication in both early-infected and chronically infected cell cultures, using nucleotide sequence-specific oligonucleotide phosphorothioates. Leither et al (1990) Proc. Natl. Acad. Sci. USA 87, 3430-3434 report inhibition in tissue culture of influenza virus replication by oligonucleotide phosphorothioates.

Oligonucleotides having artificial linkages have been shown to be resistant to degradation in vivo. For example, Shaw et al (1991) in Nucleic Acids Res. 19, 747-750, report that otherwise unmodified oligonucleotides become more resistant to nucleases in vivo when they are blocked at the 3′ end by certain capping structures and that uncapped oligonucleotide phosphorothioates are not degraded in vivo.

A detailed description of the H-phosphonate approach to synthesizing oligonucleoside phosphorothioates is provided in Agrawal and Tang (1990) Tetrahedron Letters 31, 7541-7544, the teachings of which are hereby incorporated herein by reference. Syntheses of oligonucleoside methylphosphonates, phosphorodithioates, phosphoramidates, phosphate esters, bridged phosphoramidates and bridge phosphorothioates are known in the art. See, for example, Agrawal and Goodchild (1987) Tetrahedron Letters 28, 3539; Nielsen et al (1988) Tetrahedron Letters 29, 2911; Jager et al (1988) Biochemistry 27, 7237; Uznanski et al (19-7) Tetrahedron Letters 28, 3401; Bannwarth (1988) Helv. Chim. Acta. 71, 1517; Crosstick and Vyle (1989) Tetrahedron Letters 30, 4693; Agrawal et al (1990) Proc. Natl. Acad. Sci. USA 87, 1401-1405, the teachings of which are incorporated herein by reference.

The oligonucleotides useful in the invention preferably are designed to resist degradation by endogenous nucleolytic enzymes. In vivo degradation of oligonucleotides produces oligonucleotide breakdown products of reduced length. Such breakdown products are more likely to engage in non-specific hybridization and are less likely to be effective, relative to their fall-length counterparts. Thus, it is desirable to use oligonucleotides that are resistant to degradation in the body and which are able to reach the targeted cells. The present oligonucleotides can be rendered more resistant to degradation in vivo by substituting one or more internal artificial internucleotide linkages for the native phosphodiester linkages, for example, by replacing phosphate with sulphur in the linkage. Examples of linkages that may be used include phosphorothioates, methylphosphonates, sulphone, sulphate, ketyl, phosphorodithioates, various phosphoramidates, phosphate esters, bridged phosphorothioates and bridged phosphoramidates. Such examples are illustrative, rather than limiting, since other internucleotide linkages are known in the art. See, for example, Cohen, (1990) Trends in Biotechnology. The synthesis of oligonucleotides having one or more of these linkages substituted fox the phosphodiester internucleotide linkages is well known in the art, including synthetic pathways for producing oligonucleotides having mixed internucleotide linkages.

Oligonucleotides can be made resistant to degradation by endogenous enzymes by “capping” or incorporating similar groups on the 5′ or 3′ terminal nucleotides, and which prevents RNAi degradation of the antisense strand (Martinez et al, 2002, Cell 110(5): 563-574). A reagent for capping is commercially available as Amino-Link II™ from Applied BioSystems Inc, Foster City, Calif. Methods for capping are described, for example, by Shaw et al (1991) Nucleic Acids Res. 19, 747-750 and Agrawal et al (1991) Proc. Natl. Acad. Sci. USA 88(17), 7595-7599, the teachings of which are hereby incorporated herein by reference.

A further method of making oligonucleotides resistant to nuclease attack is for them to be “self-stabilized” as described by Tang et al (1993) Nucl. Acids Res. 21, 2729-2735 incorporated herein by reference. Self-stabilized oligonucleotides have hairpin loop structures at their 3′ ends, and show increased resistance to degradation by snake venom phosphodiesterase, DNA polymerase I and fetal bovine serum. The self-stabilized region of the oligonucleotide does not interfere in hybridization with complementary nucleic acids, and pharmacokinetic and stability studies in mice have shown increased in vivo persistence of self-stabilized oligonucleotides with respect to their linear counterparts.

Alternatively or in addition, at least one nucleotide can be modified to enhance uptake of the nucleic acid molecule by a cell, or at least one nucleotide may be modified for any other purpose relating to the improvement of its activity in biological systems.

It is appreciated that if the nucleic acid molecule is to be introduced into a cell by expression from a polynucleotide or vector that encodes and expresses the nucleic acid molecule, it will usually be limited to naturally occurring nucleotides and not chemically modified nucleotides.

A second aspect of the invention provides a polynucleotide that encodes a nucleic acid molecule according to the first aspect of the invention. It will be appreciated that in this aspect, the nucleic acid molecule is one which is encodable and typically will have no chemically modified nucleotides.

A third aspect of the invention provides a vector that comprises the polynucleotide of the second aspect of the invention.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Other suitable vectors or genetic constructs are described below with respect to the administration of nucleic acid molecules to an individual.

A fourth aspect of the invention provides a host cell or stable cell line that comprises the vector of the third aspect of the invention.

The host cell cam be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast and mammalian cells, preferably vertebrate cells such as those from a mouse, rat, monkey or human fibroblastic cell line. Yeast host cells include YPH499, YPH500 and YPH501 which are generally available from Stratagene Cloning Systems, La Jolla, Calif. 92037, USA. Preferred mammalian host cells include Chinese hamster ovary (CHO) cells available from the ATCC as CCL61, NIH Swiss mouse embryo cells NIH3T3 available from the ATCC as CRL 1658, and monkey kidney-derived COS-1 cells available from the ATCC as CRL 1650. Preferred insect cells are Sf9 cells which can be transfected with baculovirus expression vectors.

By “stable” we mean that the cell-line retains its ability to express useful quantities of the nucleic aid molecule of the invention after several (e.g. 10) generations, with any decrease in the level of expression being sufficiently low not to materially affect the utility of the cell-line.

Thus, in addition to the transformed host cells themselves, the present invention also contemplates a culture of those cells, preferably a monoclonal (clonally homogeneous) culture, or a culture derived from a monoclonal culture, in a nutrient medium.

Whilst it is possible for the nucleic acid molecule, polynucleotide or vector to be administered to an individual alone, it is preferable to present it as a pharmaceutical formulation, together with one or more acceptable carriers.

A fifth aspect of the invention thus provides a pharmaceutical composition comprising the nucleic acid molecule according to the first aspect of the invention, or the polynucleotide of the second aspect, or the sector of the third aspect of the invention, and a pharmaceutically acceptable carrier, diluent or excipient.

The carrier(s) must be “acceptable” in the sense of being compatible with the compound of the invention and not deleterious to the recipients thereof.

Typically, the carriers will be water or saline which will be sterile and pyrogen free.

The nucleic acid molecule according to the first aspect of the invention, the polynucleotide of the second aspect, and the vector of the third aspect of the invention, are each encompassed by the term “compounds of the invention”.

Preferably, the formulation is a unit dosage containing a daily dose or unit, daily sub-dose or an appropriate fraction thereof, of the active ingredient.

The invention includes a pharmaceutical composition comprising the nucleic acid molecule according to the first aspect of the invention, or the polynucleotide of the second aspect, or the vector of the third aspect of the invention, and a pharmaceutically acceptable delivery system.

The deliver system may be liposomes, virosomes, microspheres or microcapsules.

The compounds of the invention may be administered systemically. Alternatively the inherent binding specificity characteristic of base pairing is enhanced by limiting the availability of the nucleic acid molecules of the invention to its intended locus in vivo, permitting lower dosages to be used and minimising systemic effects. Thus, compounds of the invention may be applied locally to achieve the desired effect. The concentration of the nucleic acid molecules of the invention at the desired locus is much higher than if they were administered systemically, and the therapeutic effect can be achieved using a significantly lower total amount. The local high concentration off the nucleic acid molecules of the invention enhances penetration of the targeted cells.

The compounds of the invention can be delivered to the locus by any means appropriate for localised administration of a drug. For example, a solution of the nucleic acid molecules or vector can be injected directly to the site or can be delivered by infusion using an infusion pump. The nucleic acid molecules or vector also can be incorporated into an implantable device which when placed adjacent to the specific site, to permit them to be released into the surrounding locus.

The compounds of the invention may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic®.

In this embodiment, the hydrogel is cooled to a liquid state and the oligonucleotides are admixed into the liquid to a concentration of about 1 mg polynucleotides per gram of hydro gel. The resulting mixture then is applied onto the surface to be treated, for example by spraying or painting during surgery or using a catheter or endoscopic procedures. As the polymer warms, it solidifies to form a gel, and the polynucleotides diffuse out of the gel into the surrounding cells over a period of time defined by the exact composition of the gel.

The compounds of the invention can be administered by means of other implants that are commercially available or described in the scientific literature, including liposomes, microcapsules and implantable devices. For example, implants made of biodegradable materials such as polyanhydrides, polyorthoesters, polylactic acid and polyglycolic acid and copolymers thereof, collagen, and protein polymers, or non-biodegradable materials such as ethylenevinyl acetate (EVAc), polyvinyl acetate, ethylene vinyl alcohol, and derivatives thereof can be used to locally deliver the compounds of the invention. They can be incorporated into the material as it is polymerised or solidified, using melt or solvent evaporation techniques, or mechanically mixed with the material. In one embodiment, compounds of the invention are mixed into or applied onto coatings for implantable devices such as dextran coated silica beads, stents, or catheters. The compounds of the invention may be administered to a patient systemically for cosmetic, therapeutic and prophylactic purposes. The compounds may be administered by any effective method, for example, parenterally (eg intravenously, subcutaneously, intramuscularly) or by oral, nasal or other means which permit them to access and circulate in the patient's bloodstream. Nucleic acid molecules or vectors administered systemically preferably are given in addition to being locally administered, but also have utility in the absence of local administration. A dosage in the range of from about 0.1 to about 10 grams per administration to an adult human generally will be effective for this purpose.

The nucleic acid molecules of the invention may be expressed from any suitable polynucleotide, genetic construct or vector as is described herein, and delivered to the patient. Although a genetic construct for delivery of the nucleic acid molecule can be DNA or RNA, it is preferred if it is DNA.

Preferably, the genetic construct or vector is adapted for delivery to a human cell.

Means and methods of introducing a genetic construct into a cell in an animal body are known in the art. For example, the constructs of the invention may be introduced into cells by any convenient method, for example methods involving retroviruses, so that the construct is inserted into the genome of the cell (see, for example, Kuriyama et al (1991) Cell Struc. and Func. 16, 503-510). For the introduction of the retrovirus into cells, it is convenient to inject directly retroviral supernatant to which 10 μg/ml Polybrene has been added. For tissue exceeding 10 mm in diameter it is appropriate to inject between 0.1 ml and 1 ml of retroviral supernatant; preferably 0.5 ml. Alternatively, as described in Culver et at (1992) Science 256, 1550-1552, cells which produce retroviruses are injected

Targeted retroviruses are also available for use in the invention; for example, sequences conferring specific binding affinities may be engineered into pre-existing viral env genes (see Miller & Vile (1995) Faseb J. 9, 190-199 for a review of this and other targeted vectors for gene therapy).

Other methods involve simple delivery of the construct into the cell for expression therein either for a limited time or, following integration into the genome, for a longer time. An example of the latter approach includes liposomes (Nässander et al (1992) Cancer Res. 52, 646-653).

For the preparation of immuno-liposomes MPB-PE (N-[4-(p-maleimidophenyl)butryl]-phosphatidylethanolamine) is synthesised according to the method of Martin & Papahadjopoulos (1982) J. Biol. Chem. 257, 286-288.

Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414).

The nucleic acid molecule or polynucleotide may also be delivered by adenovirus wherein it is present within the adenovirus particle, for example, as described below.

In an alternative method, a high-efficiency nucleic acid delivery system that uses receptor-mediated endocytosis to carry DNA macromolecules into cells is employed, for example by conjugating the iron-transport protein transferrin to polycations that bind nucleic acids.

It will be appreciated that “naked DNA” and DNA complexed with cationic and neutral lipids may also be useful in introducing the compound of the invention into cells of the individual to be treated. Non-viral approaches to gene therapy are described in Ledley (1995) Human Gene Therapy 6, 1129-1144.

Alternative targeted delivery systems are also known such as the modified adenovirus system described in WO 94/10323 wherein, typically, the DNA is carried within the adenovirus, or adenovirus-like, particle. Other suitable viruses or virus-like particles include HSV, adeno-associated virus (AAV), vaccinia and parvovirus. Thus, it will be appreciated that a further aspect of the invention provides a virus or virus-like particle comprising a compound of the invention.

A sixth aspect of the invention provides the nucleic acid molecule according to the first aspect of the invention, or the polynucleotide of the second aspect, or the vector of the third aspect of the invention for use in medicine. Thus the nucleic acid molecule or polynucleotide or vector is packaged and presented for use in medicine.

The nucleic acid molecule of the invention has a large number of potential uses. By virtue of its second domain containing a particular binding motif, the nucleic acid molecule can form a specific binding pair with a desired RNA processing or translation factor, typically a protein or an RNA molecule. By virtue of its first domain being able to form a specific binding pair with a target RNA sequence of a target RNA species, the nucleic acid molecule recruits the RNA processing or translation factor to the target RNA species having the target sequence.

Thus in a seventh aspect the invention provides a method of recruiting an RNA processing or translation factor to a target RNA species, the method comprising:

providing a nucleic acid molecule having a first domain capable of forming a first specific binding pair with a target sequence on the target RNA species, and a second domain capable of forming a second specific binding pair with an RNA processing or translation factor, and

contacting the nucleic acid molecule with the target RNA species and with the RNA processing or translation factor.

The preferences for the nucleic acid molecule in this and all subsequent aspects of the invention are as defined above in the first aspect of the invention.

The target RNA species may be any suitable target RNA species and is typically an RNA species found within a mammalian, particularly human, cell. The target RNA species may be encoded by a mammalian gene or it may be encoded by a viral gene. The target RNA species may be encoded by a common research organism such as Drosophila or C. elegans. Typically, when an RNA processing factor is recruited, the target RNA species is an unspliced (or partially unspliced) RNA. When a translation factor is recruited, the target RNA species may be fully spliced RNA. Typically, the target RNA species is one which is associated with a disease or is transcribed from a gene which is associated with a disease. The target RNA species may be an RNA molecule other than mRNA or pre-mRNA.

Typically, macromolecules such as an RNA processing or translation factor are recruited to an RNA species at which they would not be present at all or only present at low or inadequate levels, either because they do not normally bind there or because the site contains a mutation that has reduced the ability of the factor to bind.

It is appreciated that the second domain may contain a sequence binding motif that recruits a particular known RNA processing or translation factor.

The method can be performed ex vivo or in vitro, for example in a cell free assay as described herein in the Examples. The method can also be performed in a cellular system as described herein in the Examples, or in a tissue-based system, or in the body.

An eighth aspect of the invention provides the use of a nucleic acid molecule having a first domain capable of forming a first specific binding pair with a target sequence on the target RNA species, and a second domain capable of forming a second specific binding pair with an RNA processing or translation factor, in the preparation of a medicament for recruiting the RNA processing or translation factor to the target RNA species.

The invention includes the use of a nucleic acid molecule having a first domain capable of forming a first specific binding pair with a target sequence on the target RNA species, and a second domain capable of forming a second specific binding pair with an RNA processing or translation factor, for recruiting an RNA processing or translation factor to the target RNA species.

It is possible and preferred to direct a macromolecule, such as an RNA processing or translation factor, to an RNA processing or translation site on the target RNA species, by virtue of the sequence of the first domain of the nucleic acid molecule being complementary to a target region close to the specific site on the RNA species.

The invention thus includes a method of recruiting of an RNA processing or translation factor to a target sequence close to an RNA processing or translation site on the target RNA species. Typically, the formation of the first specific binding pair and the second specific binding pair recruits the RNA processing or translation factor to an RNA processing or translation site on the target RNA species to affect RNA processing or translation at said RNA processing or translation site.

The recruitment of the RNA processing or translation factor to the RNA processing or translation site on the target RNA species stimulates the reaction performed by that factor at the RNA processing or translation site. For example, as described in more detail below, an RNA splicing factor can be used to increase splicing at a splicing site or cryptic splice site, a polyadenylation factor can be used to enhance polyadenylation at a polyadenylation site, a translation factor can be used to initiate translation at a translation initiation site, and so on. Thus the invention can be used to direct RNA processing or translation to an RNA processing or translation site on the target RNA species, and to increase the level of RNA processing or translation at the RNA processing or translation site.

The invention thus includes a method of increasing the level of RNA processing or translation at a specific RNA processing or translation site.

For example, in the case of a nucleic acid molecule having a sequence motif recognised by a splicing factor, the nucleic acid molecule may stimulate incorporation of an exon that is normally excluded in a particular cell or tissue, or it may compensate for genetic damage to natural enhancer sequences in the pre-mRNA.

The present invention is unique in that trans-acting enhancers nay be tethered to the pre-mRNA substrate so that the enhancers act positively. For example, in cases of SMA the nucleic acid molecule may stimulate the inclusion of exon 7 in SMN2 transcripts through the recruitment of SR proteins (see Example 1).

The nucleic acid molecule may be considered to be a trans-acting enhancer of splicing at a specific splice site.

In an embodiment, the second domain of the nucleic acid molecule forms a specific binding pair with an RNA processing or translation factor which may be any RNA or protein that stimulates splicing activity when recruited to the RNA target species at the RNA processing site to effect RNA processing at the RNA processing site.

The target sequence of a target RNA species may be located within an exon or intron of the target RNA species. It is envisaged that, when there is genetic damage to a 5′ splice site within the 3′-most terminal nucleotides of an exon (for example the three 3′-most nucleotides) or the 5′-most terminal nucleotides of an intron (for example the eight 5′-most nucleotides), the RNA processing factors to be recruited may comprise the U1 snRNP RNA splicing factor, which plays an important role in the recognition of a 5′ splice site and the definition of an exon. In the case of mutation within an RNA cryptic splicing site, the RNA processing factors to be recruited may also comprise the U1 snRNP RNA splicing factor. Many splice site mutations are contained within a few nucleotides preceding the splice site; these are recognised primarily by U1 snRNP and tethering a good U1 binding site nearby may permit use of the correct site.

In addition, the present method may be utilised to stimulate use of the correct splice site in cases where the mutated nucleotide is not recognised by other factors.

The invention thus includes a method of increasing the level of splicing at a desired splice site, which may be a cryptic splice site, on a target RNA species, wherein the first domain of the nucleic acid molecule forms a specific binding pair with a target sequence close to the desired splice site on the RNA species, and wherein the second domain forms a specific binding pair with an RNA splicing factor.

It may be advantageous where endogenous mutant and non-mutant isogenes are present to enhance the splicing of the non-mutant form of the gene, altering the ratio of two encoded isoforms. For instance, the gene may be Ich-1 (encoding Caspase 2), for which the exclusion or inclusion of exon 9 promotes or blocks apoptosis, respectively (Wang, S. et al (1998) Cell 92(4): p 501-509, PMID: 9491891). Whereas prior art methods for blocking splicing may reduce inclusion, but could not stimulate it, the present method may be used to promote exon inclusion.

The incorporation of specific exons into transcripts can also be of therapeutic benefit in certain disease states, for example, in SMA as described in detail herein and in the examples. The involvement of alternative splicing in human disease is discussed in Caceres & Kornblihtt (Trends in Genetics, 18(4): 186-193 (2002)), incorporated herein by reference.

The invention thus includes a method of increasing the level of incorporation of a specific exon in a pre-mRNA species into a mature mRNA species, wherein the first domain of the nucleic acid molecule forms a specific binding pair with a target sequence in or around the specific exon of the pre-mRNA species, for example in the flanking introns, and wherein the second domain forms a specific binding pair with an RNA splicing factor.

Such a nucleic acid molecule is, as far as the inventors are aware, the first example of a trans-acting agent for promoting the inclusion of a specific exon in a mature mRNA molecule.

The invention further includes a method of treating a condition characterised by defective or undesirable RNA splicing in an individual, the method comprising administering to the individual a nucleic acid molecule having a first domain capable of forming a specific binding pair with a target region of a defectively spliced target RNA species and having a second domain that forms a specific binding pair with an RNA splicing factor, wherein the target region of the target RNA species is sufficiently close on the RNA species to the site of defective splicing for splicing at the site to be enhanced by the action of the splicing factor.

A condition characterised by undesirable splicing is one where an alternative splicing pattern may be preferred.

The invention also includes the use of a nucleic acid molecule having a first domain capable of forming a specific binding pair with a target region of a defectively spliced target RNA species and having a second domain that forms a specific binding pair with an RNA splicing factor, in the preparation of a medicament for treating a condition characterised by defective RNA splicing of the target RNA species, wherein the target region is sufficiently close on the RNA species to the site of defective splicing for splicing at the site to be enhanced by the action of the splicing factor.

There are approximately 30-40,000 genes in mammals that encode proteins. Of these, almost all are spliced, with an average of 9 exons and 8 introns. Thus there are in principle about 500,000 splice sites that can be targeted. Furthermore, something like half of these genes produce alternative forms of mRNA and protein, often in a tissue-specific way. Some diseases are associated with nucleotide changes in exons that disrupt splicing enhancers, and the second most common cause of all genetic disease is a single nucleotide change at a 5′ splice site. Examples of single nucleotide changes at 5′ splice sites that give rise to genetic diseases, and diseases associated with nucleotide changes in exons that disrupt splicing enhancers are given in Cartegni, L., Chew, S. L. & Krainer, A. R. (2002) Nat. Rev. Genet. 3, 285-298. This article also provides a general definition of a splice site and makes clear the range of possible different sequences of splice sites. The entire disclosure of Cartegni et al (2002) is incorporated herein by reference.

Conditions in which the invention can be of therapeutic benefit include SMA, breast cancer, Becker muscular dystrophy and β-thalasaemia.

For treating SMA, a nucleic acid molecule such as the GGA oligonucleotide described in the examples may be administered to a patient. The nucleic acid molecule may be administered by intramuscular injection, and it reaches the proximal horn of the spinal cord by retrograde transport up the motor neuron to the cell nucleus.

For example, the first domain of the nucleic acid molecule could be complementary to a region of exon 18 of BRCA1 and the second domain could contain a sequence motif (splicing enhancer sequence) that is recognised by a splicing factor, to rescue proper incorporation of exon 18 in cases where a missense mutation causes exon skipping (Liu et al (2001) Nature Genetics 27, 55-58).

For another example, the first domain of the nucleic acid molecule could be complementary to a region of exon 27 of the dystrophin gene and the second domain could contain a sequence motif (splicing enhancer sequence) that is recognised by a splicing factor, to rescue proper incorporation of this exon in cases where a mutation causes exon skipping and Becker muscular dystrophy (Shiga et al (1997) J. Clin. Inv. 100, 2204-2210).

In a further example, the first domain of the nucleic acid molecule could be complementary to the 3′ end of exon 1 the β-globin gene and the second domain could contain a 5′ splice consensus to stimulate splicing at the proper site in those cases of β-thalasaemia in which mutations in the splice site prevent its use and lead to the use of cryptic splice sites instead (Treisman et al (1983) Nature 302, 591-6.

Other diseases suitable for treatment by the compounds and methods of this invention are discussed by Cartegni et al (2002) supra, and in Caceres & Kornblihtt (2002) supra, both of which are incorporated by reference. Indeed, according to Krawczak et al (1992, Human Genetics 90: 41-54) 15% of single-base changes affect splice sites.

Thus according to a ninth aspect of the present invention, there is provided the use of a nucleic acid molecule according to the first aspect of the invention in the manufacture of a medicament for the treatment of RNA processing or translation defects of the human or animal body caused by mutations in RNA that affect binding of RNA processing or translation factors.

According to a tenth aspect of the present invention, there is provided a method of treating RNA processing or translation defects of the human or animal body caused by mutations in RNA that affect binding of RNA processing or translation factors, the method comprising administering a nucleic acid molecule according to the first aspect of the invention to an individual in need thereof.

According to an eleventh aspect of the present invention there is provided a method for the manufacture of a medicament for the treatment of RNA processing or translation defects caused by mutations in RNA that affect binding of RNA processing or translation factors, characterised in the use of a nucleic acid molecule according to the present invention.

According to a twelfth aspect of the present invention there is provided a method for the treatment of RNA processing or translation defects caused by mutations in RNA that affect binding of RNA processing or translation factors; comprising administering to a patient a medicament or pharmaceutical composition according to the present invention as described above. Medicaments can include pharmaceutically acceptable carriers, diluents or excipients (Remington's Pharmaceutical Sciences and US Pharmacopoeia, 1984, Mack Publishing Company, Easton, Pa., USA; United States Pharmacopoeia, ISBN: 1889788031). The appropriate dosage will be readily apparent to one skilled in the art (based on eg dose-response results). The medicament according to the present invention can be administered to a patient in need of the same.

Besides modulating splicing, the present invention may also be useful in affecting translation. In eukaryotes, the initiation of mRNA translation is generally thought to occur by a cap-binding/-scanning mechanism. However, some mRNA molecules are translated efficiently in the absence of a free 5′ end or cap structure, and some of these mRNA molecules contain sequences within their 5′ untranslated regions (5′ UTRs) which can directly recruit the translation machinery. Such internal ribosome entry site (IRES) elements have been found in both cellular and viral mRNA molecules. The present invention may be utilised to stimulate translation of a particular transcript by recruiting components of the ribosome or eukaryotic initiation factors, by using recently discovered short IRES sequences (or modules) which can stimulate translation (Chappell, S. A. et al (2000) Proc. Nat. Acad. Sci. USA 97(4): p 1536-1541, PMID: 10677496). These short 9nt IRES nodules are complementary to 18S rRNA sequences (nt 1132-1124) and stimulate translation either alone or synergistically as linked copies by recruiting the 40S ribosomal subunit as a first step in translation of an mRNA (Chappell, S. A. et al, Supra). Since virtually all eukaryotic mRNAs are monocistronic and capped, the ability to internally initiate traanslation might reflect the ability of a sequence to recruit ribosomes. If the recruitment of ribosomes is sufficient to enhance translation, IRES elements or modules contained within an mRNA may give that mRNA an advantage over other mRNAs which rely on a cap-dependent mode of translation initiation and scanning. Using a modified oligonucleotide wherein the first domain is complementary to a region proximal to the start codon of the transcript, and the second domain contains an IRES module or RNA sequences known to bind initiation factors (such as eIF4G and eIF3) or ribosomal components, each of which may be considered to be RNA translation factors, it may be possible to promote or stimulate the translation of that particular mRNA.

The stimulation of translation of specific transcripts can be of therapeutic benefit in certain disease states, for example, a stimulation of translation of a utrophin transgene can rescue dystrophin deficiency in mice (Rafael, J. A. et al (1998) Nat. Genet. 19(1): p 79-82, PMID: 9590295).

Thus in a thirteenth aspect, the invention provides a method of treating a condition characterised by inadequate or defective translation of an RNA species in an individual, the method comprising administering to the individual a nucleic acid molecule having a first domain capable of forming a specific binding pair with a target region of an inadequately or defective translated target RNA species and having a second domain that forms a specific binding pair with an RNA translation factor, wherein the target region of the target RNA species is sufficiently close on the RNA species to a translation initiation site for translation at the site to be enhanced by the action of the translation factor.

A fourteenth aspect of invention provides the use of a nucleic acid molecule having a first domain capable of forming a specific binding pair with a target region of an inadequately or defective translated target RNA species and having a second domain that forms a specific binding pair with an RNA translation factor, in the preparation of a medicament for treating a condition characterised by inadequate or defective translation of an RNA species, wherein the target region is sufficiently close on the RNA species to a translation initiation site for translation at the site to be enhanced by the action of the translation factor.

For example, it may be helpful to express utrophin as a substitute for dystrophin in mouse models of muscular dystrophy (Tinsley et al, 1998, Nature Medicine 4(12) :1441-1444). Utrophin has a long 5′ untranslated region (UTR), and a nucleic acid molecule having a first domain that forms a specific binding pair with a target sequence in the utrophin 5′UTR and a second domain that forms a specific binding pair with a translation factor, may be useful in increasing expression of utrophin to combat muscular dystrophy.

The present invention may also be useful in modulating polyadenylation and thus could offer potential therapeutic benefit to patients infected with retroviruses such as HIV, for example. One of the major strategies required for successful expression of the retrovirus genome is regulation of polyadenylation (poly (A)) signals contained within the long terminal repeats (LTRs)—sequences which flank the viral genome and contain the necessary signals for DNA integration. In the case of HIV-1, both the 5′ and 3′ LTRs contain poly (A) signals and the virus has evolved ways of selectively activating the poly (A) signal in the 3′ LTR, whilst suppressing use of the poly (A) signal in the 5′ LTR. This occlusion of poly (A) signal usage in the 5′ LTR is achieved through the binding of U1 snRNP to a splice site close to the poly (A) signal (Ashe, M. P. et al (2000) RNA 6: p 170-177, PMID: 10688356). To reverse this occlusion of polyadenylation by HIV-1, oligonucleotides consisting of a sequence complementary to the HIV-1 RNA sequence close to the poly (A) signal, and a tail sequence or sequences containing motifs designed to recruit polyadenylation reaction components maybe used. For example, AAUAAA sequences may be used to recruit cleavage and polyadenylation specificity factor (CPSF), which interacts with cleavage stimulatory factor (CStF), cleavage factor I (CFI), cleavage factor II and finally poly(A) polymerase (PAP) before cleavage occurs. Since cleavage and polyadenylation are linked, the free 3′ ends generated by cleaving are then rapidly polyadenylated. This stimulation of polyadenylation in the 5′ LTR could potentially decrease the expression of the HIV-1 genome.

A further aspect of the invention provides a method of enhancing polyadenylation at a desired polyadenylation site on a target RNA species, the method comprising:
providing a nucleic acid molecule having a first domain that is capable of forming a first specific binding pair with a target sequence close to the desired polyadenylation site on the target RNA species, and a second domain that is capable of foaming a first specific binding pair with an RNA polyadenylation factor, and
contacting the nucleic acid molecule with the target RNA species and with the RNA splicing factor.

The invention includes the use of a nucleic acid molecule having a first domain that is capable of forming a first specific binding pair with a target sequence close to the desired polyadenylation site on a target RNA species, and a second domain that is capable of forming a first specific binding pair with an RNA polyadenylation factor, in the preparation of a medicament for increasing the level of polyadenylation at a desired polyadenylation site on the target RNA species.

The invention includes a method of inhibiting expression of the HIV genome, the method comprising:
providing a nucleic acid molecule having a first domain that is capable of forming a first specific binding pair with the HIV-1 RNA sequence close to the poly (A) signal in the 5′ LTR, and a second domain that is capable of forming a first specific binding pair with cleavage and polyadenylation specificity factor (CPSF), and
contacting the Nucleic acid molecule with HIV and CPSF.

Therapeutically, the contacting step would typically be carried out inside a cell infected with HIV.

The invention includes the use of a nucleic acid molecule having a first domain that is capable of forming a first specific binding pair with the HIV-1 RNA sequence close to the poly (A) signal in the 5′ LTR, and a second domain that is capable of forming a first specific binding pair with by CPSF, in the preparation of medicament for inhibiting expression of the HIV genome.

Also provided is a method of effecting RNA processing or translation in an in vitro system characterised in the use of a nucleic acid molecule according to the first aspect of present invention. In vitro systems can include cell free extracts (see example 1) or cells grown in tissue culture. A nucleic acid molecule according to the present invention can be introduced to such a cell free system and RNA processing allowed to take place such that the nucleic acid molecule effects RNA processing.

It is appreciated that the nucleotide sequence of a nucleic acid molecule suitable for use in the methods of the invention, such as a nucleic acid molecule according to the first aspect of the invention, can be determined a priori from knowledge of the gene sequence, knowledge of known enhancer motifs for use in the second domain, and, depending upon the intended use, knowledge of any gene defect(s).

In a further aspect the invention provides a method of designing a nucleic acid molecule that affects RNA processing or translation at an RNA processing or translation site on a target RNA species, the method comprising:
(a) identifying the RNA processing or translation site on the target RNA species, and
(b) designing an oligonucleotide molecule comprising:
   (i) a nucleotide sequence that forms a specific binding pair with a target sequence close to the RNA processing or translation site on the target RNA species, and
   (ii) a nucleotide sequence motif that forms a specific -binding pair with an RNA processing or translation factor which affects processing or translation of the target RNA species at the RNA processing or translation site.

The invention also includes a method of producing a nucleic acid molecule for affecting RNA processing or translation at an RNA processing or translation site on a target RNA species, the method comprising designing a nucleic acid molecule as described above, and synthesizing it.

The invention further includes a method of producing a nucleic acid molecule for affecting RNA processing or translation at an RNA processing or translation site on a target RNA species, the method comprising designing a nucleic acid molecule as described above, and expressing the nucleic acid molecule from a polynucleotide encoding it.

In an embodiment, the target RNA molecule is transcribed from a defective or mutated disease gene.

The invention includes a nucleic acid molecule or oligonucleotide obtainable or obtained by any of these methods

The contents of each of the references discussed herein, including the references cited therein, are herein incorporated by reference in their entirety. Where “PMID:” reference numbers are given for publications, these are the PubMed identification numbers allocated to them by the US National Library of Medicine, from which full bibliographic information and abstract for each publication is available at www.ncbi.nlm.nih.gov.

Where “OMIM:” reference numbers are listed, they refer to the “Online Mendelian Inheritance in Man” database, which is a catalog of human genes and genetic disorders authored and edited by Dr. Victor A. McKusick and his colleagues at Johns Hopkins and elsewhere, and developed for the World Wide Web by NCBI, the National Center for Biotechnology Information. The database contains textual information, references, links to MEDLINE and sequence records in the Entrez system, and links to additional related resources at NCBI and elsewhere (www.ncbi.nlm.nih.gov/ entrez/query.fcgi?db=OMIM).

The invention will be further apparent from the following figures which show, by way of example only, embodiments of the present invention for providing positively acting RNA signals in trans.

Of the figures:

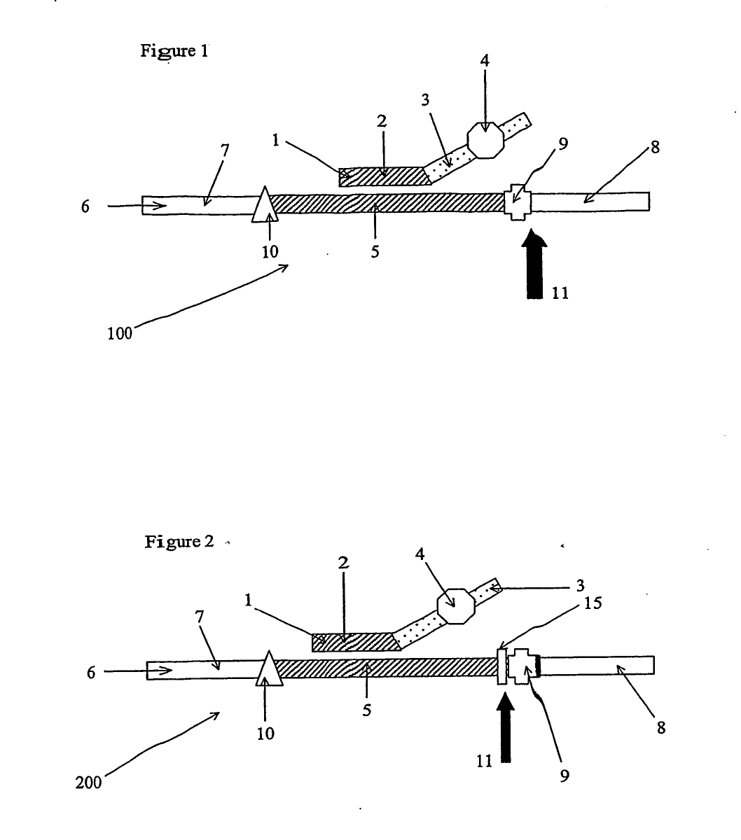

FIGS. 1 and 2 show model representations of the recruitment of RNA splicing enhancer factors according to the present invention;

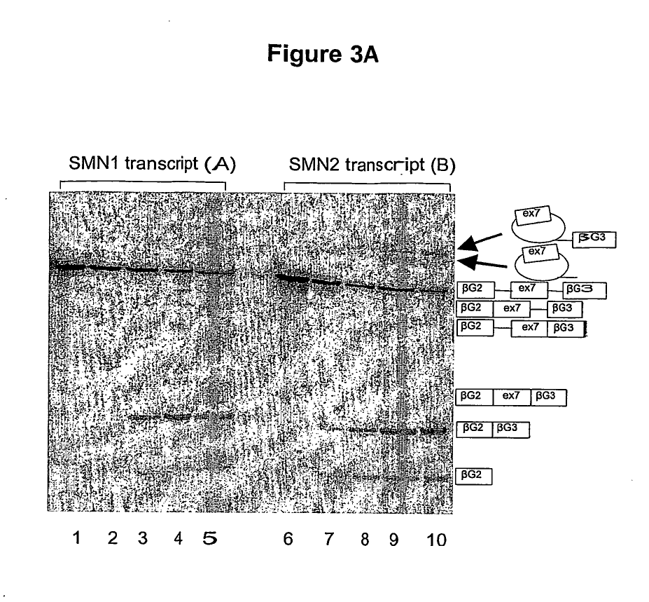

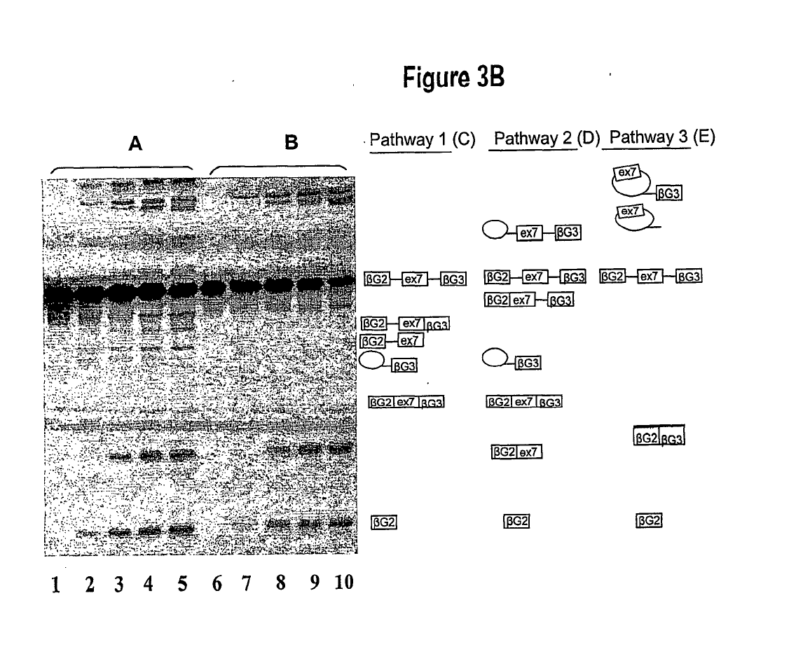

FIG. 3 shows results of an in vitro splicing assay incorporating SMN1 and SMN2 transcripts showing alternative splicing. (A) Cell-free in vitro splicing assay using [α-³²P]-labelled SMN1 transcripts (A, lanes 1-5) and SMN2 transcripts (B, lanes 6-10). Transcripts were incubated at 30° C. for 0, 30 minutes, 1, 2 or 3 hours, before termination of the reactions. The reactions were then ethanol precipitated and mixed with 5 μl F-dyes and 3 μl was loaded and fractionated on a 5% denaturing polyacrylamide gel. Lanes 1-5 and 6-10 represent different time points: lanes, 1, 6: 0 minutes; lanes 2, 7: 30 minutes; lanes 3, 8: 1 hour; lanes 4, 9: 2 hours and lanes 5, 10: 3 hours. (B) Timed assay of SMN1 transcripts (A, lanes 1-5) and SMN2 transcripts (B, lanes 6-10) using a transcript containing a longer exon 3. This allows the band corresponding to exon 2 spliced to exon 3 to be separated from the splice intermediate of exon 2 spliced to exon 7. In the previous figure these two bands run together on the gel. Lanes 1-5 and 6-10 represent different time points: lanes 1, 6: 0 minutes; lanes 2, 7: 30 minutes; lanes 3, 8: 1 hour; lanes 4, 9: 2 hours and lanes 5, 10: 3 hours. This figure also shows the three different splicing pathways that occur (C, D, and E)—pathways C and D promote exon 7 inclusion while pathway E skips exon 7;

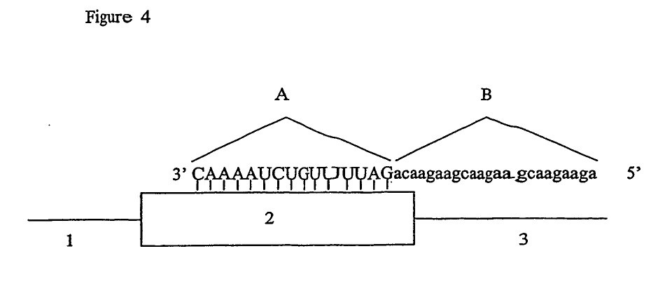

FIG. 4 shows a diagrammatical representation (not to scale) of a tailed oligonucleotide bound to SMN2 exon 7 (2). Intron 6 (1), and Intron 7 (3) are also shown. The complementary RNA sequence of the oligonucleotide (A) is in upper case, while the tail region containing sequences that mimic exonic splicing enhancers are in lower case (B). The oligonucleotide binds via a complementary region (A) to the first part of SMN2 exon 7 (2), the non-complementary tail region (B) remains unbound and is thus available to bind to splicing proteins present in the in vitro splicing reaction mix;

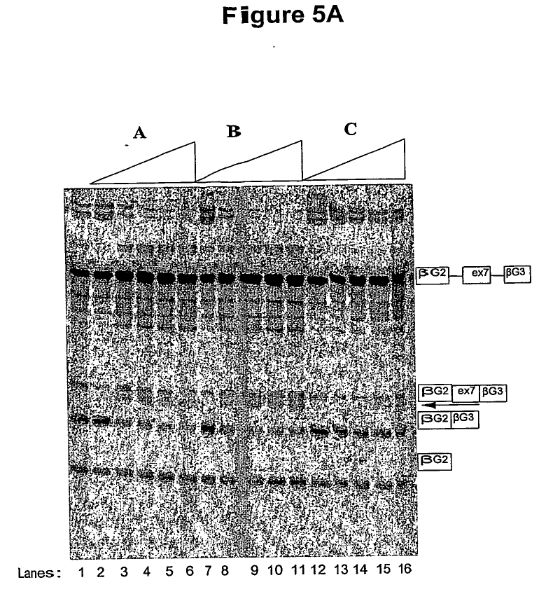

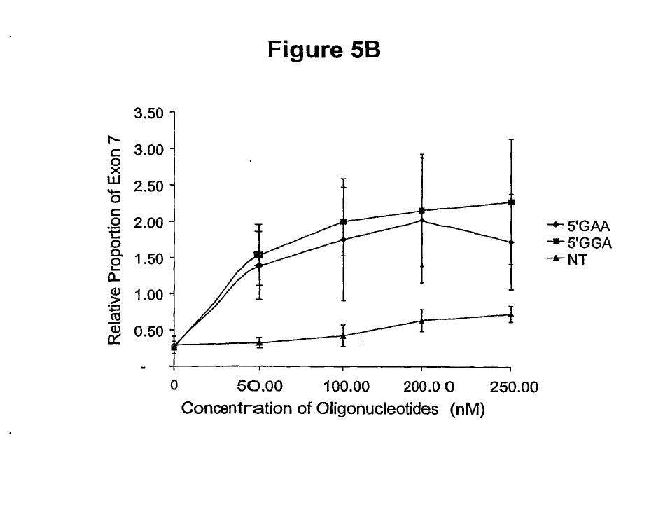

FIG. 5 shows tailed 5′GAA and 5′GGA oligonucleotides promote exon 7 inclusion. (A) Cell-free in vitro splicing assay using [α-³²P]-labelled SMN2 transcripts combined with oligonucleotide 5′GAA (lanes 2-6, A), oligonucleotide 5′GGA (lanes 7-11, B) and oligonucleotide NT (no tail region) (lanes 12-16, C). The oligonucleotides were either not included in the splicing reactions (lanes 2, 7, and 12), or incorporated at 50 nM (lanes 3, 8, and 13), 100 nM (lanes 4, 9, and 14), 200 nM (lanes 5, 10, and 15) or 250 nM (lanes 6, 11, and 16), respectively. The splicing reactions were allowed to proceed for 3 hours before termination of the reactions. The reactions were then ethanol precipitated and mixed with 5 μl F-dyes and 3 μl was loaded on a 5% denaturing polyacrylamide gel. The SMN1 transcript was included in lane 1. The lariat produced from intron 2 is shown with an arrow. (B) Graph showing relative proportion of exon 7 inclusion (y-axis) relative to the SMN1 level of splicing with increasing concentrations of oligonucleotides (0-250 nM, x-axis). Data points marked with diamonds correspond to the 5′ GAA oligonucleotide, data points marked with squares correspond to the 5′ GGA oligonucleotide, and data points marked with triangles correspond to the NT oligonucleotide. The SMN1 transcript was included in all gels as an internal control enabling successive gels to be directly correlated. The results of three experiments were combined to produce this data. Standard deviations varied from 0.03 to 0.86;

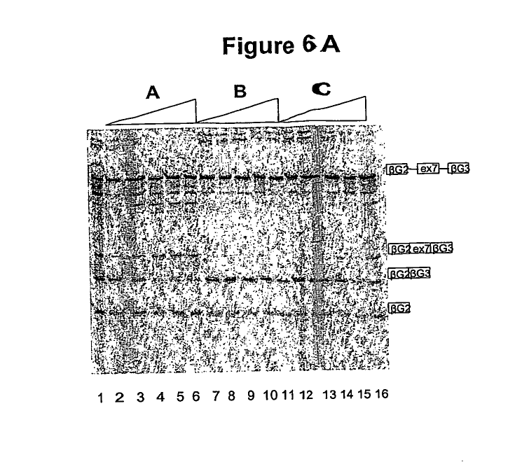

FIG. 6 shows the application of 5′PTB and 5′A1 oligonucleotides to SMN2 transcripts. (A) Cell-free in vitro splicing assay using [α-³²P]-labelled SMN2 transcripts combined with oligonucleotide 5′GAA (lanes 2-6, A), oligonucleotide 5′PTB (lanes 7-11, B) and oligonucleotide 5′A1(lanes 12-16, C). The oligonucleotides were either not included in the splicing reactions (lanes 2, 7, and 12), or incorporated at 50 nM (lanes 3, 8, and 13), 100 nM (lanes 4, 9, and 1 4), 200 nM (lanes 5, 1 O, and 15) or 250 nM (lanes 6, 11, and 16), respectively. The splicing reactions were allowed to proceed for 3 hours before termination of the reactions. The reactions were then ethanol precipitated and mixed with 5 μl F-dyes and 3 μl was loaded on a 5% denaturing polyacrylamide gel. The SMN1 transcript was included in lane 1. (B-D): Graphs showing the percentage of RNA (y-axis) in the initial pre-mRNA transcript (data points marked with diamonds), the exon 7 included product (data points marked with squares) and the skipped product (data points marked with triangles) at increasing concentrations (x-axis) of 5′GGA oligonucleotide (FIG. 6B), 5′PTB oligonucleotide (FIG. 6C), and 5′A1 oligonucleotide (FIG. 6D). The products have been corrected for the numbers of labelled radionucleotides in each form of RNA. These graphs were plotted from a single experiment, but the results were reproducible in at least three different experiments;

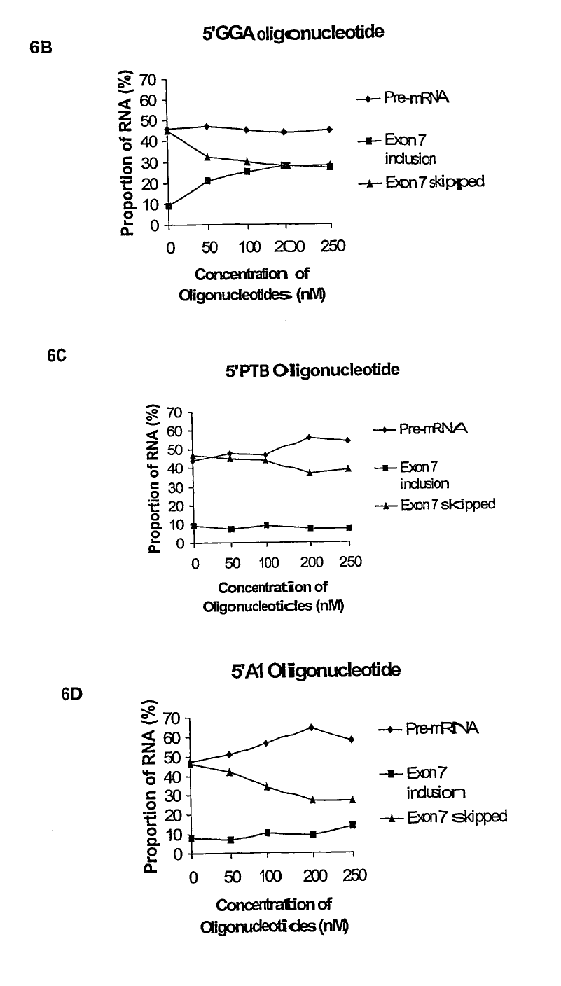

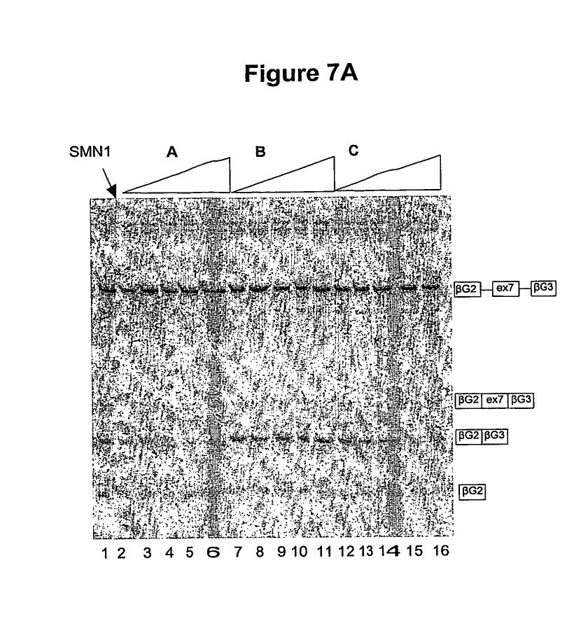

FIG. 7 shows the application of ‘tail only’ oligonucleotides to the SMN2 transcript in the in vitro system. (A) Cell-free in vitro splicing assay using [α-³²P]-labelled SMN2 transcripts combined with oligonucleotide 5′GAA-TO (lanes 2-6, A), oligonucleotide 5′PTB-TO (lanes 7-11, B) and oligonucleotide 5′A1 -TO (lanes 12-16, C). The oligonucleotides were either not included in the splicing reactions (lanes 2, 7, and 12), or incorporated at 50 nM (lanes 3, 8, and 13), 100 nM (lanes 4, 9, and 14), 200 nM (lanes 5, 10, and 15) or 250 nM (lanes 6, 11, and 16), respectively. The splicing reactions were allowed to proceed for 3 hours before termination of the reactions. The reactions were then ethanol precipitated and mixed with 5 μl F-dyes and 3 μl was loaded on a 5% denaturing polyacrylamide gel. The SMN1 transcript was included in lane 1. (B-D) Graphs showing the percentage of RNA (y-axis) in the initial pre-mRNA transcript (data points marked with diamonds), the exon 7 included product (data points marked with squares) and the skipped product (data points marked with triangles) at increasing concentrations (x-axis) of 5′GAA-TO oligonucleotide (FIG. 7B), 5′PTB-TO oligonucleotide (FIG. 7C), and 5′A1-TO oligonucleotide (FIG. 7D). The products have -been corrected for the numbers of labelled radionucleotides in each form of RNA; and

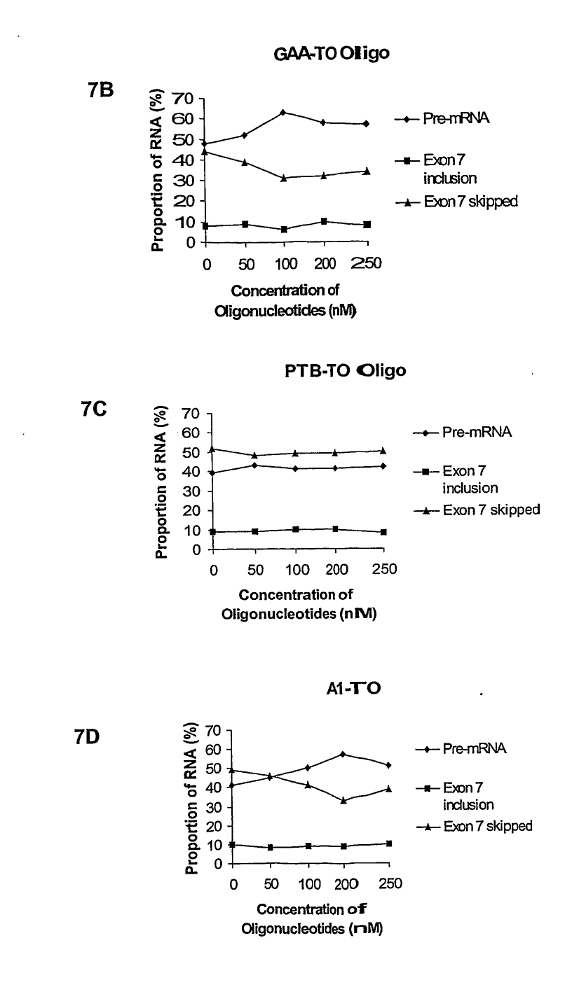

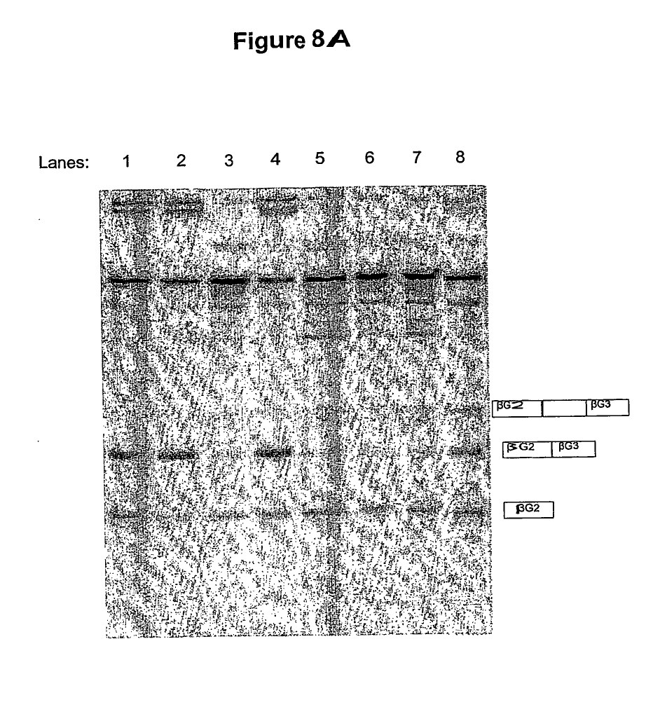

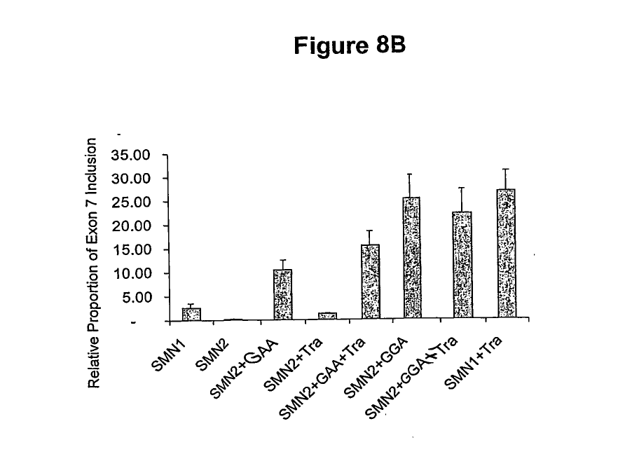

FIG. 8 shows enrichment of splicing reactions with Recombinant Tra2 proteins. (A) 5% polyacrylamide gel showing the effect or SMN splicing of enriching the HeLa cell extract with recombinant Tra2 protein, both in the absence and presence of the antisense oligonucleotides. The proteins were added at a final concentration of 1 pM and preincubated for 10 minutes at 30° C. with the HeLa cell extract. The first two lanes show the SMN1 and SMN2 transcripts, respectively, without the presence of oligonucleotides or added SR proteins. Lane 3: SMN2+GAA oligo; lane 4: SMN2+Tra2; lane 5: SMN2+GAA+Tra2; lane 6: SMN2+GGA oligo; lane 7: SMN2+GGA+Tra2; lane 8: SMN1+Tra2. (B) Bar chart showing the results of enriching the HeLa cell extract with recombinant Tra2. X-axis: relative proportion of exon 7 inclusion, y-axis (bars 1 -8) corresponds to lanes 1-8 in FIG. 8A.

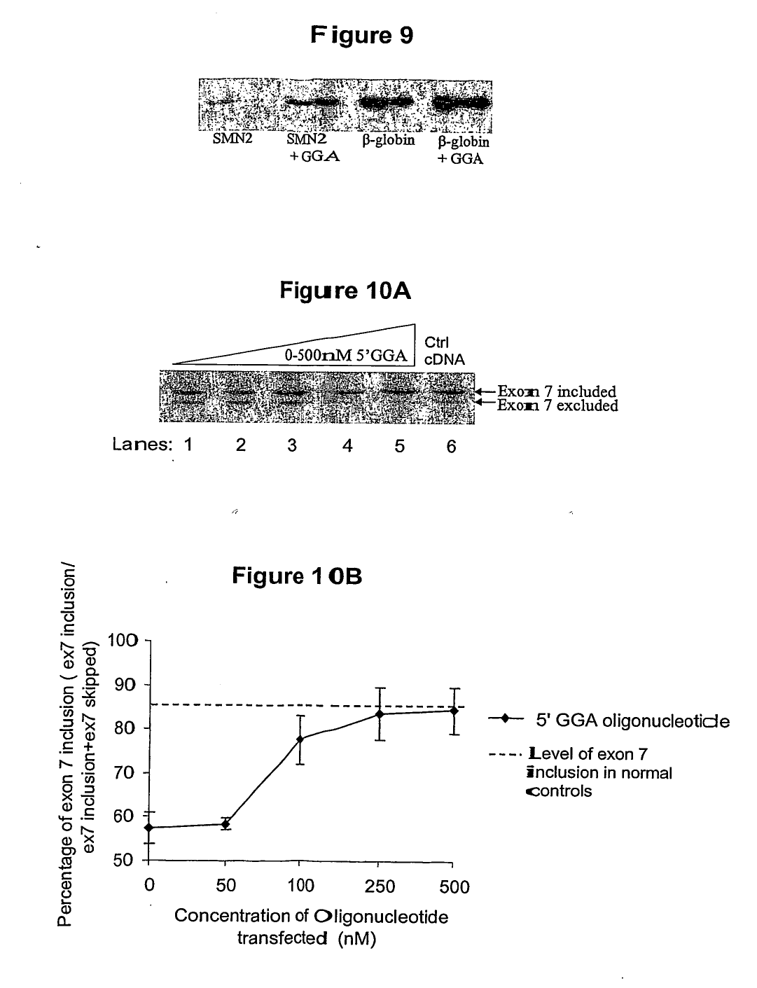

FIG. 9 shows recruitment of SF2/ASF to SMN2 exon 7 by the 5′ GGA oligonucleotide. Biotinylated RNA (SMN2 exon 7 or b-globin, as indicated) was bound to streptavidin beads and incubated in nuclear extract. The proteins associated with the RNA were separated by SDS/PAGE, and SF2/ASF was detected by Western blotting. Lanes “+GGA” indicate that the RNA was incubated with the GGA oligonucleotide before addition to the beads.

FIG. 10 shows the transfection of type I SMA patient fibroblasts. (a) Denaturing polyacrylamide gel (6%) showing the results of a semiquantitative RT-PCR, using primers situated in exons 6 and 8 of the SMN gene, was carried out on cDNA from cells transfected with increasing concentrations of the 5′ GGA oligonucleotide. Lane 1, untransfected cells; lane 2, 50 nM oligonucleotide-transfected; lane 3, 100 -nM oligonucleotide-transfected; lane 4, 250 nM oligonucleotide-transfected; lane 5, 500 nM oligonucleotide-transfected; lane 6, normal control. (b) Graph showing the percentage of exon 7 inclusion in the transcripts derived from the 5′ GGA transfected cells. The results of three different transfection experiments were combined to produce the graph. The readings were corrected for the amounts of labeled radionucleotides, and the percentage of exon 7 inclusion was calculated by exon 7 inclusion mRNA/total mRNA. The horizontal dashed line represents the percentage of exon 7 inclusion obtained in control fibroblasts.

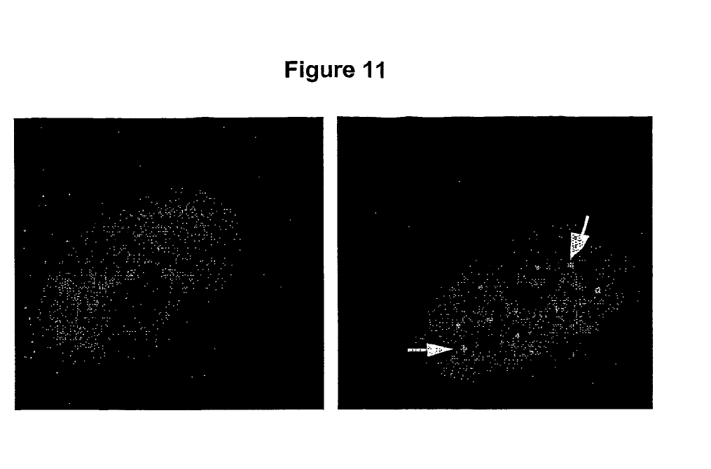

FIG. 11 shows the restoration of gems in SMA patient fibroblasts. Images show untransfected (a) and transfected (b) SMA type I fibroblasts. 4′,6-Dianidino-2-phenylindole staining highlights the nuclei in blue, and the white arrows indicate the gems (red dots in nucleus). Untransfected cells show 2-3% of their nuclei containing gems, whereas transfected cells show 13% gem-positive nuclei.

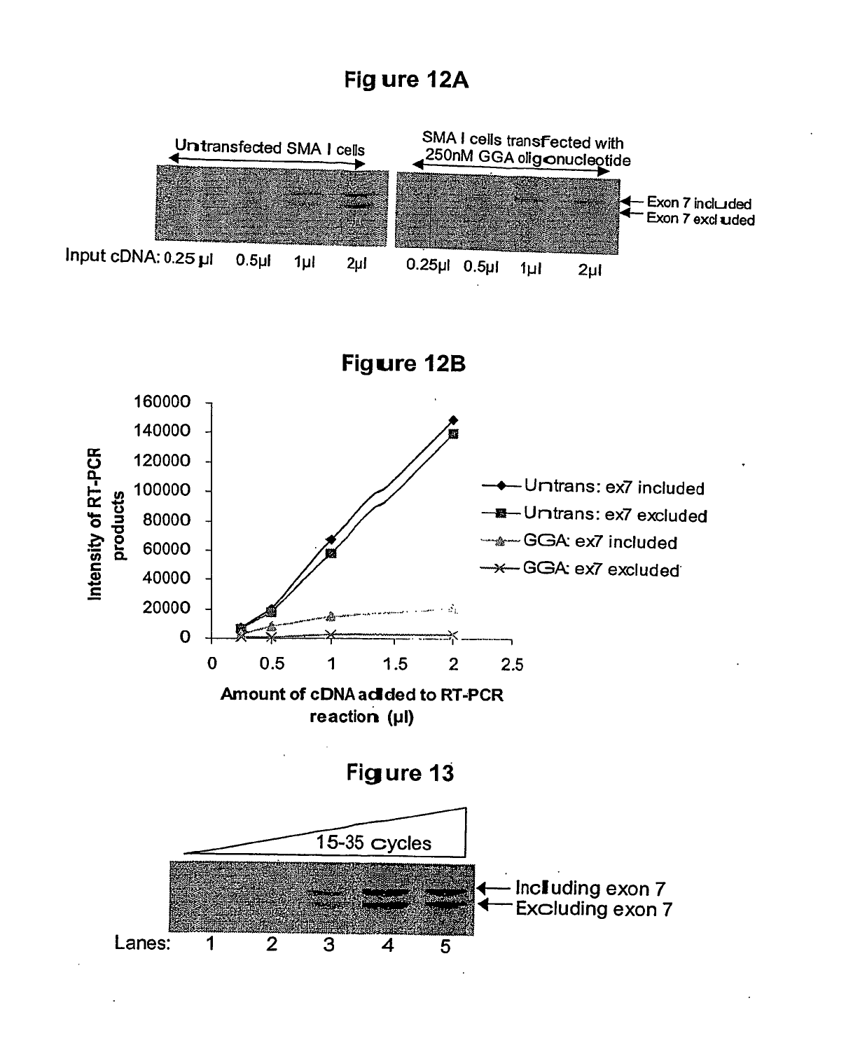

FIG. 12a shows the results of RT-PCR reactions carried out on two different samples of cDNA, one derived from RNA from untransfected SMA patient cells and one from patient cells transfected with 250 nM 5′GGA oligonucleotide. The RT-PCRs were performed using an 8-fold range of concentrations of input cDNA (0.25-2 μl) as indicated on the figure. Primers in exons 6 and 8 of the SMN gene were used and the RT-PCR was carried out for 20 cycles in the presence of [α-³²p] dATP. FIG. 12b is a graph showing the variation of RT-PCR product intensity with the Volume of input cDNA. The data labels ‘untrans’ and ‘GGA’ refers to cDNA derived from untransfected SMA patient cells and cells transfected with 250 nM 5′GGA oligonucleotide respectively. The intensities of the exon 7 included and ex on 7 excluded products have been quantified and plotted in order to show that a linear relationship exists between these two isoforms regardless of the amount of input cDNA. Signal intensities were quantified and standard deviations of 0.7 and 5.1 were obtained for the percentages of exon 7 inclusion which were 52.6 and 83.3 in the untransfected and transfected samples respectively.

FIG. 13 is a gel showing RT-PCR products resulting from 15-35 cycles. Lane 1: 15 cycles, lane 2: 20 cycles, lane 3: 25 cycles, lane 4: 30 cycles and lane 5: 35 cycles. The proportions of the signal in the upper band in lanes 2, 3, 4 and 5 are respectively 61%, 59%, 55% and 54%. The signal in lane 1 is unmeasurable.

In a general embodiment (100) of the present invention (FIG. 1), the splicing of a particular exon (5) of an pre-mRNA transcript (6) is stimulated by attachment of a modified oligonucleotide (1) with exogenous enhancer sequences to the exon (5). Exon (5) is defined at its 5′ end by a splice site (10) adjacent to intron (7), and at its 3′ end by splice site (9) adjacent to intron (8). The modified oligonucleotide (1) has a first exon-annealing domain (2) and a second domain (3) with a sequence known to act as a splicing enhancer. After entry into the target cell, the first domain (1) anneals to the complementary sequence of the exon (5). The second domain (3) recruits enhancer proteins (4), causing the level of splicing (indicated by arrow 11) at the splicing site (9) to increase. This may be done to alter expression in specific tissues, or to counteract mutations in or around the exon that have led to it being excluded during splicing.

In a second general embodiment (200) of the present invention (FIG. 2), a modified oligonucleotide (1) is tethered close to either a cryptic splice site (15) and/or a normal splice site (9) such that the recruiting domain (3) of the oligonucleotide (1) behaves as though it were part of the target pre-mRNA transcript (6) itself. Modified oligonucleotide (1) has a first exon-annealing domain (2), complementary to a sequence on exon (5), and a second domain (3) with a sequence that recruits the splicing protein U1 snRNP (4). Tethering of U1 snRNP (4) to a location near to the cryptic splice site (15) and/or the splice site (9) activates either or both sites, causing an increasing in splicing (indicated by arrow 11).

Experiments

### EXAMPLE 1

The following example relates to spinal muscular atrophy and details the use of a novel strategy to modify the splicing of SMN2 that is, in principle, widely applicable to exons that are included at sub-optimal levels. Oligonucleotides have been designed that, while they are complementary to the target exon, do not block reactions at their binding sites like conventional antisense RNA. Instead, the oligonucleotides incorporate a non-complementary ‘tail’ consisting of sequences that mimic exonic splicing enhancers. We show here that these tailed oligonucleotides induce the inclusion of SMN2 exon 7 with high efficiency in a cell-free splicing assay. We also show that this approach was successful in vivo: the proportion of exon 7 inclusion in mRNA from the endogenous SMN2 gene was increased in fibroblasts from patients with SMA to match the levels seen in control fibroblasts, and the formation of gems, intranuclear structures containing SMN, was partially rescued. This novel approach has potential therapeutic implications for SMA and other conditions, and also more generally for the study and modification of splicing regulation and RNA processing.

Methods

β-Globin/SMN Constructs for Use in Cell-free Splicing Assays

Rabbit β-Globin exon 2, intron 2 and the beginning of exon 3 -was amplified by PCR using the novel primers BGEX2F and BGEX3R (see Table 1 for sequences). This PCR product was cloned into the TOPO cloning vector (Invitrogen). SMN1 and SMN2 exons 7 and flanking regions were PCR amplified from previously sequenced clones. Novel primers SALRIIF and SALSMNR were used to create Sal I sites. Sal I digested PCR products were then cloned into the similarly digested TOPO/β-Globin vector created previously. The SalI site was situated within the intronic region between the two β-globin exons such that SMN1 or SMN2 exon 7 and intronic regions were situated between the two β-globin exons.

Site-directed Mutagenesis of SMN Constructs

The stop codon at the end of SMN exon 7 was altered in order to allow read-through of the β-globin/SMN constructs. This was achieved by site-directed mutagenesis using the sequenced SMN constructs obtained above as templates. The vectors were amplified with reverse complementary primers SMN7XF and SMN7XR containing a base pair deletion and a nucleotide change. The PCR was carried out using Pfu turbo polymerase (Hybaid), with the following cycles: 95° C. for 30 seconds, then 12 cycles of 95° C. for 30 seconds, 55° C. for 1 minute and 68° C. for 8 minutes. After successful amplification, the mixture was transformed and the positive clones sequenced.

In-vitro Transcription Mix

Novel primers with the forward primer (T7BGEX2F) incorporating the T7 promoter sequence, and a reverse primer (BGEX3R), situated in β-globin exon 3, were used to amplify SMN exon 7 and flanking β-globin exons from the β-globin/SMN1 and β-globin/SMN2 constructs, resulting in an 800 bp product. 100 ng of the PCR products were then combined in an in vitro transcription mix and the transcripts labelled with [α-³²P]-GTP at 37° C. for 3-4 hours. 10 μl Fdyes were then added and the mixture was fractionated on a 5% polyacrylamide gel at 30 W for approximately 1.5 hours.

Visualisation of Transcription Products

The gel plates were separated and the gel exposed to Biomax X-ray film (Kodak) for 1-5 minutes before developing. The transcript bands were excised from the gel, placed in SDS lysis buffer and incubated at 4° C. overnight.

In vitro Splicing

The radiolabelled transcripts were ethanol precipitated and resuspended in 20 μl TE containing 0.1% RNase inhibitor (RNasin, Promega). A stock splicing mix was made containing 0.5 μl 100 mM ATP; 4 μl 0.5 M Creatine Phosphate; 4 μl 80 mM MgCl₂; 2 gl HEPES buffer, pH 7.5; 0.3 μl RNasin and 17 μl 13% Polyvinyl alcohol. Finally, 40 μl HeLa nuclear extract and 20 μl DKCL/DGlu full buffer (Eperon, I. C. et al (2000) Mol. Cell. Biol. 20: p 8303-8318, PMID: 11046128) were added to the stock splicing mix. 0.5 μl of each labelled transcript was then aliquoted into PCR tubes and 4.5 μl splicing mix was added. A timed assay was then carried out with reactions at 0, 30 minutes, 1, 2 and 3 hours, such that an increase in the spliced products can be seen over time. The splicing reactions were placed at 30° C. and removed to the −80° C. freezers at each relevant time point. 50 μl Proteinase K stop mix was added to the thawed reactions and placed at 37° C. for 10 minutes. The samples were then ethanol precipitated and resuspended in 10 μl F-dyes and 3 μl loaded and fractionated on a 5% denaturing polyacrylamide gel (as described previously). The gel was then fixed and dried in a gel drier and exposed for 3-5 hours to a phosphor screen. ImageQuant software (Biorad) was used to quantify the products in experiments using the SMN/β-globin transcripts. The levels of radioactivity were not corrected to allow for the different numbers of labelled nucleotides in the RNA products.

Splicing Protein Binding Motif-tailed Antisense Oligonucleotides

A series of 10 tailed antisense oligonucleotides were designed (see table 2 for sequences). They all contained both 2′-O-methyl and phosphorothioate modifications and were obtained from EuroGentec, France. These oligonucleotides were complementary to the 5′ end of exon 7 and in addition contained tails designed to recruit various proteins. Two of them (5′ GAA and 3′GAA) contained an identical tail situated on either the 5′ or 3′ end of the oligonucleotide, designed to initially establish the most effective position for the tail. The 5′GAA oligonucleotide was designed to bind to hTra2-β1, while the 5′GGA oligonucleotide was designed to recruit SF2/ASF. Other tailed oligonucleotides (5′ PTB and 5′ A 1) were designed to recruit polypyrimidine tract binding protein (PTB) and hnRNP A1, respectively. Since these proteins do not stimulate splicing, the 5′ PTB and 5′ A1 oligonucleotides served as useful negative controls. Other control oligonucleotides contained either no tail (NT), or consisted of a scrambled sequence (Scram). Three oligonucleotides consisting of the tail regions only of 5′ GAA, 5′ PTB and 5′ A1 were also synthesized and used as controls. The oligonucleotides were incorporated to final concentrations of 0, 50, 100, 200 and 250 nM and pre-incubated for 10 minutes with the SMN2 transcript at 30° C. prior to the addition of the splicing mix. The reactions were allowed to proceed for 3 hours at 30° C. All experiments were repeated in triplicate and the relative abundance of the spliced products was normalized against SMN1 readings (included as an internal control) and the mean values plotted on a graph.

Enrichment of HeLa Cell Extract with hTra2β Protein

Recombinant GST-Tra2, was expressed along with the SR protein kinase 1 (SRPK1) in E. coli BL21 (DE3). The protein was purified by affinity chromatography using glutathione-agarose beads by incubation in 0.5 M KCl at 30° C., using standard protocols. GST-tagged Tra2 recombinant protein was preincubated at 30° C. for 10 minutes in the HeLa cell extract splicing mix prior to the addition of the transcripts. The protein had a final concentration of 1 pM and was added to reactions either with or without the GGA or GAA oligonucleotides (used at 250 nM final concentration)

SF2/ASF Binding Assays

Biotinylated SMN2 RNA was produced by transcription of a PCR product that comprised exon 7 with an additional 12-nt 3′ extension that provided a strong U1 small nuclear ribonuclear protein binding site. RNA (10 pmol) was incubated with 10 pmol of the 5′ GGA oligonucleotide at 30° C. for 10 min in Dglu buffer. The RNA was added to 10 ml of streptavidin agarose beads (Sigma) prewashed in 20 mM Hepes (pH 8), 150 mM NaCl, and 0.05% Triton X-100. After 2 h at 4° C., the beads were washed three times by centrifugation in the same buffer for 2 minutes in a microfuge at 3,000 rpm (850×g). A standard splicing reaction mixture (74 ml) containing HeLa cell nuclear extract was added. After incubation at 30C for 10 min, the beads were washed three times as above but without centrifugation, and the proteins were eluted and separated by 12% SDS/PAGE. The separated proteins were transferred to nitrocellulose membrane and detected with anti-SF2 antibody and protein A/G peroxidase (Pierce). Chemiluminescence was detected on film, and the intensity was measured by using a Kodak EDAS 290 camera system. Quantification of the image used OPTIQUANT (Packard). For the rabbit β-globin control, the biotinylated RNA contained exon 2, a truncated version of intron 2, and 50 nucleotides of exon 3, amounting to ≈380 nucleotides.

Cell Culture and Transfections

SV-40-transformed human SMA type I fibroblast cell lines derived from two different patients were grown in DMEM containing 10% (vol/vol) FCS and 2 mM glutamine. Cells were plated at 3×10⁴ cells per well in 24-well plates 18-24 h before transfection. Each well was treated with 50, 100, 250, or 500 nM oligonucleotide complexed with jetPEI (Qbiogene, Nottingham, UK) transfection reagent. After a 5-h incubation, 10% FCS media was added. Transfections using the 5′ GGA oligonucleotides were carried out three to five times, whereas single transfections were performed with control oligonucleotides (5′ Al and NT).

Quantitative RT-PCR of SMN Transcripts

Total RNA was extracted 24 h after transfection by means of the Qiagen RNeasy kit. First-strand cDNA synthesis was carried out with Superscript II reverse transcriptase (Invitrogen). The endogenous SMN transcripts were amplified by using the primers 541C618 and 541C1120 situated in exons 6 and 8, respectively, of the SMN gene (20). The PCR consisted of 20 cycles and was carried out in the presence of [α-³²P]dATP. The resulting PCR products were boiled and run on a 6% denaturing polyacrylamide gel, and IMAGEQUANT software was used for quantification. To exclude the possibility of unequal efficiency of cDNA amplification arising during RT-PCR because of differing concentrations of input cDNA, an 8-fold range of concentrations of starting cDNA was tested by using cDNA from the patient-derived fibroblast cell line. The ratios of the PCR products derived from mRNA produced by inclusion and skipping of exon 7 were constant across the range. Without transfection, the percentage of spliced mRNA that had incorporated the exon was 52.6 (SD=0.7). After transfection with 250 nM GGA oligonucleotide, the percentage was 83.3 (SD=5.1). There was no detectable effect of starting cDNA concentrations. The validity of the PCR amplification was also checked by cycle curves. The amplification efficiency of the shorter isoform (excluding exon 7) was calculated to be higher by a factor of ≈1.02 (18). Thus, the maximum possible distortion of the ratio over 20 cycles is ≈1.5-fold, whereas the ratio of includedy excluded isoforms detected by RT-PCR changes after transfection from 1.1 to 4.9, a difference of >4-fold. We conclude that RT-PCR is an insignificant source of errors.

Immunohistochemistry

The SMA fibroblast cell lines were plated on collagen-coated (Nutacon, Leimuiden, The Netherlands) coverslips in 24-well plates, and the oligonucleotide transfections were performed the following day. Twenty-four hours after transfection, immunofluorescent staining was carried out as described (21). The anti-SMN mAb MANSMA2 (22) was diluted 1:100, and a fluorophore-labeled donkey anti-mouse IgG diluted 1:2,000 was used to visualize the anti-SMN Ab staining. The coverslips were mounted with 4′,6-diamidino-2-phenylindole (DAPI; Vector Laboratories), and the cells were visualized on a Leica confocal microscope by using the ×100 objective.

Results

SMN1 and SMN2 Transcripts Replicate the Alternative Splicing of Endogenous Transcripts within an in vitro System

In vitro splicing assays of pre-mRNA containing SMN1 or SMN2 exon 7 and flanking intronic regions can be seen in FIG. 3A, where the SMN sequences were set between exons 2 and 3 of rabbit β-globin. Splicing reactions with three exons are relatively difficult to interpret because there are three possible pathways for splicing: skipping, inclusion via splicing of intron 1 before intron 2, and inclusion via splicing of intron 2 before intron 1. To assist in assigning the bands, the splicing experiments were repeated with a longer 3′-most exon (FIG. 3B). Most of the bands could be assigned by direct side-by-side comparisons of the two reactions, with the exception of the faint bands containing a single intron in a lariat. Based on these assignments, we were able to identify the mRNA derived by skipping and inclusion of exon 7. The faint band below the skipped mRNA ie β-Globin exons 2 and 3 spliced together, in FIG. 3B is an intermediate in the inclusion pathway that proceeds via splicing of intron 1 before intron 2. With the shorter transcripts, this product co-migrates with the skipped product, but its level is very much less (FIG. 3B). Thus, we used the shorter transcript for most experiments because it spliced with greater efficiency and ignored the minimal contribution from the intermediate.

The splicing efficiencies of both the longer and shorter transcripts recapitulated the splicing pattern of endogenous human SMN genes. When comparing the intensities of the bands representing exon 7 inclusion with the total amount of spliced product, for the shorter transcript (FIG. 3A), we found that SMN1 exon 7 inclusion was on average 3.5-fold higher (24.6%±6.3 mean inclusion) than that of SMN2 (7%±0.74 mean inclusion). These figures have not been corrected for the numbers of labelled radionucleotides in each form of RNA, allowing the values to be correlated directly with inspection of the bands seen on the images. The readings varied between 19.0-32.30% for the SMN1 and 6.5-8.1% for the SMN2 transcripts. Exon inclusion appeared to result from both possible pathways, but the majority of transcripts seemed to follow the route in which intron 1 was removed first.

Modified Tiled Antisense Oligonucleotides Increase Exon 7 Inclusion within the SMN2 Transcript

Antisense oligonucleotides were designed that were complementary to SMN exon 7 and contained additional non-complementary sequences (tails) that were predicted to recruit splicing enhancer factors (see FIG. 4). Two of these contained identical tails of GAA repeats on either the 5′ or the 3′ side of the oligonucleotide. These oligonucleotides were designed to initially establish the most effective position for the tail. The choice of the GAA sequence was based on the known ability of hTra2β to bind to GAA sequences as well as published experimental evidence that Tra2β protein is able to bind to the SMN exon and, when transfected into cells, to enhance its inclusion. Another oligonucleotide contained a 5′ GGA tail, GGAGGA being a subset of the sequences shown by functional SELEX to mediate the effects of the SR protein, SF2/ASF. Furthermore, (GGA) repeats are a feature of a number of enhancers, including human tropomyosin TPM3. Control oligonucleotides contained either no tail, or consisted of a scrambled sequence. The oligonucleotides were incubated with the pre-mRNA substrate and then mixed with a splicing reaction mixture. Both the 5′GAA and 5′GGA oligonucleotides reduced the level of the SMN2 exon 7 skipped product even at the lowest concentration (50 nM), and they increased the level of the mRNA product including exon 7 (FIG. 5A). The increase in inclusion was relatively weak with the GAA oligonucleotide (compare lanes 2, without oligonucleotide, and 3) but robust with the GGA oligonucleotide (lanes 8-11). In contrast, the NT (no tail) oligonucleotide had very little effect. The relative proportion of inclusion in SMN2 rose with both the GAA and GGA oligonucleotides to levels higher than for SMN 1 (FIG. 5B), although with the GAA oligonucleotide this was largely caused by the inhibition of skipping. Other experiments (not shown) showed that the 5′ GAA tail was more effective than the 3′ GAA tail, and therefore all other tailed oligonucleotides carried 5′ tails. A comparison of the bands produced by splicing in the presence of the 5′GGA oligonucleotide with the pattern of SMN1 splicing shows that the oligonucleotide produces a relatively high level of bands corresponding to pathway 1 linear intermediates containing the first intron (cf. FIG. 3B). This suggests that pathway 1 is promoted, ie, that removal of intron 2 is accelerated and that the pathway intermediates accumulate because splicing of intron 1 is limiting. Another relatively abundant band can be seen underneath the inclusion mRNA in FIG. 5A, which may represent intron 2, but this has not been formally verified.

The Increase in SMN2 Exon 7 Inclusion at Low Concentrations of Oligonucleotide Requires an Enhancer-like Sequence and Base-pairing to Exon 7

To test whether the effects seen were a specific consequence of attaching the enhancer-like sequence to SMN2 exon 7, other tailed oligonucleotides mere designed that were predicted to recruit inhibitors of splicing such as PTB or hnRNP A1. In contrast to the 5′GGA oligonucleotide, neither 5′PTB or 5′Al oligonucleotides caused a significant increase in the level of exon 7 inclusion at the lower concentrations (FIG. 6A and B-D). However, whereas the 5′PTB oligo bad no effect at any concentration (FIG. 6C), the higher concentrations of 5′A1 produced a marked decrease in the level of skipped mRNA and a very slight increase in inclusion (FIG. 6D). This is likely to be the result of sequestration of hnRNP A1 by un-annealed oligonucleotide.

To test this, and to establish whether the enhancement of exon 7 inclusion by the 5′GAA oligonucleotide resulted from binding to the pre-mRNA, oligonucleotides consisting of the tail regions only of 5′ GAA, 5′ PTB and 5′A1 were synthesized. The “tail-only” oligonucleotides were incorporated in the SMN2 splicing reactions as previously described (FIG. 7A and B-D) The results showed that the 5′ A1-TO produced a concentration-dependent decrease in skipping akin to that of the 5′ Al oligonucleotide (FIG. 7D), consistent with the hypothesis that the A1 oligonucleotide was sequestering ‘free’ hnRNP A1 proteins irrespective of its action on SMN2 exon 7. The 5′GAA-TO oligonucleotide produced a decrease in skipping but it had no effect on exon 7 inclusion (FIG. 7B).

To determine whether base pairing of the oligonucleotide to the substrate was required, we tested an oligonucleotide containing the GGA repeat ESE sequence in which the sequence complementary to SMN2 exon 7 was reversed (called RevGGA). This oligonucleotide produced only a small and nonsaturable change up to concentrations of 1 mm, irrespective of the presence of the ESE sequence (data not shown).

Effects of Recombinant Tra2 Protein on SMN1 but not SMN2

Recombinant GST-Tra2 protein was added to slicing reactions to determine whether the attachment of the tailed oligonucleotides would increase responsiveness to Tra2β. The results (FIG. 8A and B) showed that GST-Tra2β stimulated splicing of SMN1, but not SMN2, even though the putative binding site for Tra2β is present in SMN2 exon 7 and it responds in vivo. The oligonucleotides stimulated exon 7 inclusion as usual, such that the level of inclusion at least matched that of SMN1, but the protein supplement bad little effect. We conclude that Tra2β binding does limit the efficiency of splicing of SMN1, but that the level of binding of SF2/ASF is also likely to be low for SN2. The oligonucleotides may bind Tra2β and other activating proteins efficiently, even in the absence of supplements but the level of SF2/ASF binding at the site of the C-T transition remains an additional barrier.

5′ GGA Oligonucleotide Mediates Binding of SF2/ASF

The increased use of the exon produced by annealing to 5′ GGA should be associated with recruitment of splicing proteins known to bind ESE sequences. One such protein is SF2/ASF, which is both limiting for SMN2 exon 7 (Cartegni & Krainer (2002) Nat. Genet. 30, 377-384) and able to bind GGA repeat sequences (Liu H. X. et al (1998) Genes Dev. 12, 1998-2012). Thus, we tested whether there was increased recruitment of SF2/ASF to SMN2 exon 7 in the presence of the 5′ GGA oligonucleotide. Biotinylated SMN2 exon 7 was incubated with the oligonucleotide, retained on beads, and then incubated in nuclear extract. Bound SF2/ASF was detected after washing by Western blotting. In three separate experiments, the binding rose by 150-280% when the 5′ GGA oligonucleotide was present (FIG. 9), whereas, in a parallel reaction with β-globin, binding rose by just 50%. We conclude that the 5′ GGA oligonucleotide does mediate an increase in the binding of at least one protein that might be expected to bind to an ESE.

Increase in Exon 7 Inclusion within the Endogenous SMN2 Gene.

To verify that our in vitro data were reproducible in an in vivo system, the tailed antisense oligonucleotides were transfected into SMA type I patient fibroblasts at various concentrations between 50 and 500 nM. By using a semiquantitative RT-PCR to analyze the splicing pattern of the 5′ GGA oligonucleotide transfected cells, a clear dose-dependent increase in exon 7 inclusion could be seen 24 h after transfection (FIG. 10a). This increase in exon 7 inclusion changed from 57% exon 7 inclusion in untransfected cells to 84% in cells transfected with 500 nM 5′ GGA oligonucleotide. This increase matched the level of exon 7 inclusion seen in control fibroblasts (FIG. 10b). The same effect was seen in both SMA patient cell lines. These results strengthen our hypothesis that the tailed oligonucleotides are capable of acting as ESE sequences in trans to create a positive effect on splicing of the SMN2 gene. The transfection efficiency of the oligonucleotides was investigated by means of a fluorescently tagged oligonucleotide of similar length to the oligonucleotides used here. An effective transfection efficiency of 90% was calculated.

To investigate the specificity of the above results, the 5′ A1 and NT oligonucleotides were also transfected into the SMA type I fibroblasts and the splicing patterns were examined. Both the 5′ A1 and NT oligonucleotide produced little or no effect on exon 7 inclusion (data not shown), indicating a clear necessity for an appropriate ESE sequence.

Increased SMN Protein Expression in SMA Type I Fibroblasts

The physiological significance of the increased exon 7 inclusion in vivo was examined by the analysis of gems. These are intranuclear structures in which SMN protein accumulates. Gem numbers in fibroblasts have been shown to correlate with phenotypic severity and are thus an indication of the amount of SMN protein present (Coovert D. D. et al, (1997) Hum. Mol. Genes 6, 1205-1214). Our control fibroblasts showed gem staining in 52% of the nuclei, with three to six gems per nucleus. In the SMA type I fibroblasts used, gems were observed in only 2-3% of nuclei, and each positive nucleus contained only a single gem. After transfection of the SMA fibroblasts, the number of positive nuclei rose to 13% and there was a very striking increase in the number of gems per nucleus, which rose to WT levels of three to six (FIG. 11). The number of gem-positive nuclei we observed was intermediate between that found in type I SMA and in carrier parent fibroblast cell lines (25% gem-positive nuclei) (Coovert et al).

To show that the results of the above RT-PCR reactions do not alter according to varying amounts of input cDNA (first-strand cDNA synthesis carried out with Superscript II reverse transcriptase on total cellular RNA) we carried out experiments using different concentrations of the same cDNA samples. The intensity of the bands produced at each concentration was then quantified as well as the percentage of exon 7 inclusion at each point. The results of these experiments are shown in FIG. 12a and 12b. The intensities of the exon 7 included and exon 7 excluded products show that a linear relationship exists between these two isoforms regardless of the amount of input cDNA.

As described above, the RT-PCR reactions were carried out at 20 cycles. However, to use PCR to measure isoform concentrations, it was necessary to demonstrate that the ratio of the exon 7 included/excluded products obtained in the RT-PCR reactions was not affected significantly by different cycle efficiencies. To do this a single PCR reaction was set up and aliquots were removed at 15, 20, 25, 30 and 35 cycles. The percentage of exon 7 inclusion was then quantified at each point. The results are shown in FIG. 13.

The proportions of the signal from the exon 7 included band after 20, 25, 30 and 35 cycles (lanes 2, 3, 4 and 5) are respectively 61%, 59%, 55% and 54%. The apparent small change in the ratio might indicate that the two bands were not being amplified with equal efficiency. This is addressed as described below.

The intensity of the bands increases by about the same factor between lanes 2 and 3 and between lanes 3 and 4. The intensities in lanes 4 and 5 were similar. We infer that amplification was approximately exponential between cycles 20 and 30. The equation describing the amplification of molecules (or signal intensities) in 2 bands was described by Eperon, I. C., & Krainer, A. R. (1994) in RNA Processing—A Practical Approach, vol I, (eds. Higgins, S. J. & Hames, B. D., IRL Press, Oxford, pp. 57-101) previously as: Log[na/Nnb]=log[N0a/N0b]+n log[effa/effb],

Where Nna=number of PCR-derived molecules (or signal) of sequence or isoform a at cycle n, N0a=number of PCR-derived molecules (or signal) of sequence a before amplification starts, i.e., after the first cycle of TCR in which single-stranded RT products are rendered double-stranded, Nnb and N0b are the corresponding values for sequence or isoform b, Effa and effb are the efficiencies of amplification of sequences a and b, where the effa is Nna/N(n−1)a during the exponential phase of amplification.

From our results, we conclude that effa/effb is 1.02, where a is the shorter isoform (excluding exon 7). Thus, over 20 cycles the ratio of N0a/N0b will change by a factor of approximately 1.6. This will have a small effect on the comparison if 2 samples (transfected and untransfected cells) are amplified exponentially for the same number of cycles. In each case, the signal will overstate the proportion of the shorter isoform. Thus the 54% upper band could be corrected to 65%, and 84% to 89%, where the percentages represent 100[b/(b+a)] (data from FIG. 10).

The corrections would actually increase the apparent success of the method, in that the corrected original proportion was 35% and that the transfected oligonucleotide had converted ⅔ of it, leaving it at 11%.

The greatest possible distortion introduced by the slightly different amplification efficiencies would be a case in which the GGA-transfected mRNA population is so abundant that there is no PCR amplification; correction would alter the untransfected %b to 65% and the real %b for the transfected cells would be 84%. In other words, even in this extreme case, there is a very clear effect of transfection. As it happens, the extreme case is quite impossible to envisage because (a) RNA extracted from transfected cells is usually less abundant and (b) 20 cycles is nowhere near the endpoint for the exponential phase.

Discussion

The two SMN genes are ˜99% similar (Lefebvre, S. et al (1995) Cell 80, 155-165). A single nucleotide difference in exon 7 results in the different splicing characteristics of the SMN1 and SMN2 genes. The SMN2 gene generates a smaller proportion of full-length RNA transcripts and a low level of SMN protein that only partially compensates for the lack of SMN1-derived protein. Correcting the deficient splicing of the SMN2 gene should increase SMN protein production and provide a therapeutic benefit to patients with SMA. Alteration of SMN2 splicing has been attempted by various investigators, some of whom have used inhibitory antisense oligonucleotides (Lim, S. R. & Hertel, K. J. (2001) J. Biol. Chem. 276, 45476-45483), but the magnitude and specificity of effects have so far been very low, making their possible therapeutic use unlikely.

ESE sequences are predominantly found in exons flanked by weak splice sites (Fairbrother, W. G. et al, (2002) Science 9, 1007-1013). In one model for their effects, they are bound by SR or other proteins that promote spliceosome formation, aiding the recognition of nearby splice sites and activating splicing (Zhu, J. & Krainer, A. R. (2000) Genes Dev. 14, 3166-3178; Graveley, B. R. (2000) RNA 6, 1197-1211; Blencowe, B. J. (2000) Trends Biochem. Sci. 25, 106-110; Graveley, B. R. et al, (2001) RNA 7, 806-818; and Guth, S. et al, (2001) Mol. Cell. Biol. 21, 7673-7681.).

The present inventors have devised a novel strategy that takes advantage of an antisense oligonucleotide approach but, in contrast to the normal use of such oligonucleotides as physical obstructions of a reaction at a target site or as mediators of RNase H degradation, we have used these oligonucleotides to attach potent enhancer sequences to the SMN exon, which then activate it. The oligonucleotides use exon 7 as a docking site and the unbound tail region sequesters SR proteins to the immediate vicinity of SMN2 exon 7. As SR proteins function in a concentration dependent manner, by increasing the local concentration of SR proteins surrounding exon 7, its inclusion in the final transcript should be increased.

In our in vitro model we have characterised a series of oligonucleotides with tails aimed at recruiting hTra23 and SF2/ASF, with PTB and hnRNP A1 proteins as controls. We show a specific increase in the proportion of exon 7 inclusion in the SMN2 mRNA when using the 5′ GGA (aimed at recruiting SF2/ASF) and 5′ GAA (hTra2β) oligonucleotides (FIG. 5A). However, in the case of the 5′ GAA oligonucleotide, the effect was largely accounted for by a decrease in the level of skipped mRNA. In both cases, the proportion of exon 7 inclusion was increased by concentrations as low as 40 nM, and it reached a plateau at concentration exceeding 250 nM. A closer examination showed that the level of exon 7 inclusion was almost maximal at the lowest concentrations of oligonucleotide tested and that the highest concentrations caused a decline in the exon-skipped product. The probable explanation for this is that an excess of the oligonucleotide depletes enhancer-binding factors from the nuclear extract. This reduces the efficiency of all splicing events (including skipping), but the oligonucleotide bound to exon 7 allows that exon to compete effectively, permitting exon 7 splicing to continue with relatively little impediment.

The maximum level of exon 7 incorporation achieved with SMN2 was seen with the 5′GGA oligonucleotide, which reproducibly increased the level to approximately the same as that seen with SMN1 or even higher. It has been shown that the C-T change between SMN1 and SMN2 in exon 7 caused the loss of an SF2/ASF binding site. Indeed, analysis of the 5′ GGA oligonucleotide with an “ESE finder program” (Cartegni, L. and Krainer, A. R., Supra) revealed that the oligonucleotide possessed high scores for three overlapping putative SF2/ASF binding motif, which were not identified in the 5′ GAA oligonucleotide, confirming the crucial role of the SF2/ASF binding domain in the inclusion of exon 7 (Cartegni, L. and Krainer, A. R., Supra).

A separate mechanism may underlie the effect on exon 7 inclusion of the 5′ GAA oligonucleotide, which is supposed to recruit hTra2-β1 protein. Enrichment of HeLa cell extract with recombinant Tra2 protein in combination with 5′ GAA and 5′ GGA oligonucleotides resulted in a specific increase in SMN2 exon 7 inclusion only when the 5′ GAA oligonucleotide was present, indicating that GAA repeat motifs rather than GGA are more effective in Tra2 binding. Enrichment of recombinant Tra2 proteins to both the SMN1 and SMN2 transcripts in the absence of any nucleotides did not result in increase SMN2 exon 7 incorporation; surprisingly, however, Tra2 dramatically increased exon 7 inclusion in the SMN1 spliced products (FIG. 8). This was unexpected because the proposed binding site of hTra2-β1 is identical in both the SMN1 and SMN2 genes. Various mechanisms could explain these findings; a possibility is a different secondary structure and accessibility of the binding sites of hTra2-β1. Alternatively it is possible that hTra2-β1 necessitates efficient SF2/ASF binding (as is the case with SMN1 but not SMN2), in order to promote exon 7 inclusion. It has long been accepted that the alternative splicing occurs as a result of a cumulative effect of many different splicing proteins acting both co-operatively and antagonistically with one another to regulate splicing. Indeed, another SR protein SRp30c, is capable of altering SMN2 splicing, but only through co-operation with HTra2-β1.

The addition of hTra2-β to the nuclear extract stimulates inclusion in SMN1, but has relatively little effect on SMN2 (FIG. 8A, compare lanes 1 & 8 with lanes 2 & 4), consistent with a limiting level of SF2/ASF binding. However-, it also has little effect on SMN2 in the presence of either the 5′GGA or 5′GAA oligonucleotide. This suggests that there may be other factors limiting even further improvements in efficiency. However, achieving an efficiency matching that of SMN1 is in itself potentially of value.

A modest increase in exon 7 incorporation was also observed when using the 5′ A1 oligonucleotide, while no effect of the 5′ PTB oligonucleotide was noticed (FIG. 6A and B-D). It was suspected that the binding of hnRNP A1 would inhibit exon incorporation, since there are several examples of exonic splicing silencers whose effect is mediated by hnRNP1. A likely mechanism is that hnRNP A1 and SF2/ASF compete for binding to the pre-mRNA, even though they have different high affinity binding sites, hnRNP A1 binding being nucleated by its high affinity sites and its propagation being limited by SF2/ASF. The absence of a high affinity site for SF2/ASF in SMN2 exon 7, caused by the C-T change at position 6, may mean that the exon is already swathed in hnRNP A1 and the effect of the oligonucleotide may be merely that un-annealed oligonucleotide titrates out some of the hnRNP A1. However, the results indicate that there is very little increase in exon 7 incorporation, and the major effect is a decrease in the level of skipped mRNA. This may indicate that (unsurprisingly) the oligonucleotide also sequesters some splicing activators.

The above results show that the use of tailed oligonucleotides containing SR binding motifs is an effective approach for controlling splicing efficiency. We have shown that the 5′GGA tailed antisense oligonucleotide results in very significant changes in the relative proportions of sipping and inclusion of exon 7 within SMN2 both in vitro and in vivo. This technique has been applied to spinal muscular atrophy, a common genetic disorder and results show that the 5′ GGA and 5′ GAA antisense oligonucleotides result in very significant inclusion of exon 7 within SMN2 at levels that are comparable to or higher than those of endogenous SMN1. The level of exon 7 incorporation obtained with the 5′GGA oligonucleotide in our in vitro experiments reached approximately that seen with the SMN1 transcripts, whereas the proportion of full-length mRNA produced from SMN2 genes in patient fibroblasts reached that of control fibroblasts at oligonucleotide concentrations as low as 250 nM. Furthermore, a significant increase in the number of gems was observed, indicating a partial restoration of full-length fully functional SMN. However, because gems were not completely restored to the levels found in control individuals, it is possible that the overall level of expression from the SMN2 gene is still limiting. These results are encouraging, because the level of gem restoration in a patient with type I SMA was similar to those observed in patients with a milder form of SMA, indicating that these levels are likely to be functional. Gene therapy strategies aimed at correcting the alternative splicing present in the SMN2 gene resulting in increased SMN production thus represent a promising means of providing therapeutic benefit to SMA patients.

The use of these antisense oligonucleotides to enhance expression of latent exons may ultimately be of therapeutic use. Various phosphorothioate oligonucleotides have reached phases I and II in clinical trials for the treatment of viral infections and cancer (Galderisi, U. et al (1:999) J. Cell. Physiol. 181, 251-257). In particular, a drug which is composed of a phosphorothioate oligonucleotide, designed to inhibit human cytomegalovirus replication, has been licensed recently (Galderisi et al, 1999).

Approximately 15% of point mutations identified produce splicing abnormalities resulting in increased exon inclusion or exclusion ultimately culminating in genetic disease (Blencowe, B. J. (2000) Trends Biochem. Sci. 25, 106-110). Our novel approach of using “tailed” oligonucleotides to alter splicing thus represents a promising new therapeutic approach not only for SMA, but for a variety of genetic disorders.

In therapeutic practice, systemic or generalized administration of SR or SR-related proteins might have a detrimental effect because of their action on multiple genes, as suggested by the toxicity observed in the experiments to produce stable transfectants expressing SR proteins (Andreassi, C. et al. (2001) Hum. Mol. Genet. 10, 284-1-2849). Instead, the method we describe here should allow specific exons to be activated at very low concentrations of oligonucleotide, especially when the issue of transport across the blood-brain barrier is resolved. The method may also have practical benefits for research in that the ability to induce incorporation of latent exons in vivo might be useful in studies of splicing mechanisms or the functions of protein isoforms.

TABLE 1
Primers used for cloning

| | Seq | |
|---|---|---|
| Name | ID No. | Sequence (5′-3′) |
| BGEX2F | 1 | GGGCTGCTGGTTGTCTACCCA |
| BGEX2F | 2 | AACTTACCTGCCAAAAATGATGAGACA |
| SALRIIF | 3 | ATTCATGTTATATGGTCGACAGACTATCAACTTAAT |
| | | TTCTG |
| SALSMNR | 4 | GTTAGCAGAGTCGACGTGATATAAAATGGCATATC |
| SMN7XF | 5 | CATTCCTTAAATCGGAGTAAGTCTGC |
| SMN7XR | 6 | GCAGACTTACTCCTGATTTAAGGAATG |
| T7BGEX2F | 7 | AAATTAATACGACTCACTATAGGGCTGCTGGTTGTC |
| | | TACCCA |

TABLE 2
Modified tailed oligonucleotides

| | Seq | |
|---|---|---|
| Name | ID No. | Sequence (5′-3′) |
| 5′ GAA | 8 | asgsasasgsaacgaagaacgaagaacaGo |
| | | AoUosUosUosUosGosUoCoUoAoAos |
| | | AosAosCo |
| 3′ GAA | 9 | GosAosUosUosUosUoGoUoCoUsoAos |
| | | AosAosAosCoagaagaacgaagaacgaa |
| | | sgsasascsa |
| 5′ GGA | 10 | asgsgsasgsgacggaggacggaggacaGo |
| | | AoUosUosUosUosGosUoCoUoAoAosAos |
| | | AosCo |
| 5′ PTB | 11 | asuscsususuccucuuuccucuuuccaGo |
| | | AoUosUosUosUosGosUoCoUoAoAosAos |
| | | AosCo |
| 5′ A1 | 12 | asusasgsgsgcaggcuagggcaggccaGo |
| | | AoUosUosUosUosGosUoCoUoAoAosAos |
| | | AosCo |
| NT (no tail) | 13 | GosAosUosUosUosUoGoUoCoUosAos |
| | | AosAosAosCo |
| Scram | 14 | oAosCosCosCosUosGoUoCoUoUosAos |
| | | GosGosUos |
| RevGGA | 15 | asgsgsasgsgacggaggacggaggacacgo |
| | | sAosAosAosUoCoUoGoUoUoUoUosAos |
| | | GosUosUosU |

In these sequences, the tail region (second domain) is given in lowercase and the complementary (antisense) RNA sequence (first domain) is in uppercase.

The letters “o” and “s” refer to 2′-O-methyl and phosphorothioate chemical groups, respectively.

1. A nucleic acid molecule comprising first and second domains, said first domain being capable of forming a first specific binding pair with a target sequence of a target RNA species, said second domain consisting of a sequence which forms a second specific binding pair with at least one RNA processing or translation factor.

2. A nucleic acid molecule according to claim 1, wherein said first domain of said nucleic acid molecule attaches to said target sequence of said RNA target species by means of complementary base pairing.

3. A nucleic acid molecule according to claim 1, wherein said second domain forms a second specific binding pair with an RNA processing or translation factor selected from the group consisting of: RNA molecules, RNA structural molecules, RNA stability molecules, RNA-protein complexes, splicing factors, polyadenylation factors, transcription factors, and translation factors, and combinations thereof.

4. A nucleic acid molecule according to claim 1, wherein said second domain forms a second specific binding pair with an RNA processing factor which is any RNA, RNA-protein complex or protein that stimulates splicing activity.

5. A nucleic acid molecule according to claim 1, wherein said second domain forms a second specific binding pair with an RNA processing factor selected from the group consisting of: SR proteins, SR-related proteins, hnRNP proteins, STAR proteins and CELF proteins.

6. A nucleic acid molecule according to claim 1, wherein said second domain forms a second specific binding pair with an RNA processing factor selected from the UsnRNP group of RNA splicing factors.

7. A nucleic acid molecule according to claim 6, wherein said second domain forms a second specific binding pair with U1, U2 or U6 snRNP.

8. A nucleic acid molecule according to claim 1, wherein said second domain forms a second specific binding pair with an RNA translation factor selected from an initiation factor, such as eIF4G and eIF3, or a ribosomal component.

9. A nucleic acid molecule according to claim 1, wherein formation of the first specific binding pair and the second specific binding pair recruits the RNA processing or translation factor to an RNA processing or translation site on the RNA target species to effect RNA processing or translation at said RNA processing or translation site.

10. A nucleic acid molecule according to claim 1, wherein the first domain is capable of forming a first specific binding pair with the target sequence on the RNA target species within 1,000 nucleotides of an RNA processing or translation site on the RNA target species.

11. A nucleic acid molecule according to claim 10 wherein the first domain is capable of forming a first specific binding pair with the target sequence on the RNA target species within 100 nucleotides of an RNA processing or translation site on the RNA target species.

12. A nucleic acid molecule according to claim 9, wherein the RNA processing or translation site on the RNA target species is selected from an RNA splicing site, a cryptic RNA splicing site, a polyadenylation site and a translation initiation site.

13. A nucleic acid molecule according to claim 9, wherein the RNA processing or translation site on the RNA target species is mutated.

14. A nucleic acid molecule according to claim 9, wherein a further site on the target RNA species is mutated, wherein the further site contributes to a protein or RNA-protein assembly required for processing or translation at the RNA processing or translation site.

15. A nucleic acid molecule according to claim 1, wherein said nucleic acid molecule comprises at least one modified nucleotide.

16. A nucleic acid molecule according to claim 15, wherein said at least one modified nucleotide is chemically modified to enhance stability or uptake by a cell.

17. A nucleic acid molecule according to claim 15, wherein said at least one modified nucleotide is selected from the group consisting of a 2′-O-methyl derivative of RNA, a phosphothiorate modification, a morpholino modification, a phosphoroamidate modification, a peptide nucleic acid derivative of RNA, and a linked nucleic acid derivative of RNA.

18. A polynucleotide that encodes the nucleic acid molecule according to claim 1.

19. A vector that comprises the polynucleotide of claim 18.

20. A host cell or stable cell line that comprises the vector of claim 19.

21. A pharmaceutical composition comprising the nucleic acid molecule according to claim 1, or the polynucleotide of claim 18, or the vector of claim 19, and a pharmaceutically acceptable carrier, diluent or excipient, or in a pharmaceutically acceptable delivery system.

22. (canceled)

23. A method of recruiting an RNA processing or translation factor to a target RNA species, the method comprising:
providing a nucleic acid molecule having a first domain capable of forming a first specific binding pair with a target sequence on the target RNA species, and a second domain capable of forming a second specific binding pair with an RNA processing or translation factor, and
contacting the nucleic acid molecule with the target RNA species and with the RNA processing or translation factor.

24. (canceled)

25. (canceled)

26. A method or a use according to claim 23, wherein formation of the first specific binding pair and the second specific binding pair recruits the RNA processing or translation factor to an RNA processing or translation site on the target RNA species to effect RNA processing or translation at said RNA processing or translation site.

27. A method or a use according to claim 23, wherein the target sequence is within 1,000 nucleotides of an RNA processing or translation site on the RNA target species.

28. A method according to claim 27 wherein the target sequence is within 100 nucleotides of an RNA processing or translation site on the RNA target species.

29. A method or a use according to claim 23 wherein the RNA processing or translation factor is selected from the group consisting of: RNA molecules, RNA-protein complexes, RNA structural molecules, RNA stability molecules, splicing factors, polyadenylation factors, transcription factors, and translation factors, and combinations thereof.

30. A method or a use according to claim 23 for increasing the level of splicing at a specific splice site on a target RNA species, wherein the first domain of the nucleic acid molecule forms a specific binding pair with a target sequence close to the specific splice site on the RNA species, and wherein the second domain forms a specific binding pair with an RNA splicing factor.

31. A method according to claim 30 wherein the specific splice site is a cryptic splice site or a mutated splice site.

32. A method according to claim 23 for increasing the level of incorporation of a specific exon in a pre-mRNA species into a mature mRNA species, wherein the first domain of the nucleic acid molecule forms a specific binding pair with a target sequence in the specific exon of the pre-mRNA species, and wherein the second domain forms a specific binding pair with an RNA splicing factor.

33. A method or a use according to claim 30 or 32 wherein the RNA splicing factor is selected from the group consisting of: SR proteins, SR-related proteins, and hnRNP proteins, CELF proteins, STAR proteins, and any RNA, RNA-protein complex or protein that stimulates splicing activity.

34. A method according to claim 23 which is performed in an in vitro cell-free system.

35. A method according to claim 23 which is performed in an ex vivo cellular system.

36. A method according to claim 23 which is performed in an ex vivo tissue-based system.

37. A method according to claim 23 which is performed in vivo in the human or animal body.

38. A method of treating a condition characterised by defective or undesirable RNA splicing in an individual, the method comprising administering to the individual a nucleic acid molecule having a first domain capable of forming a specific binding pair with a target region of a defectively spliced target RNA species and having a second domain that forms a specific binding pair with an RNA splicing factor, wherein the target region of the target RNA species is sufficiently close on the RNA species to the site of defective or undesirable RNA splicing for splicing at the site to be enhanced by the action of the splicing factor.

39. (canceled)

40. A method according to claim 38 wherein the RNA splicing factor is selected from the group consisting of: SR proteins, SR-related proteins, and hnRNP proteins, and any RNA or protein that stimulates splicing activity.

41. A method according to claim 38 wherein the defective RNA splicing is caused by a mutation at the site of defective splicing.

42. A method according to claim 38 wherein enhanced exonic incorporation is desirable at the site of undesirable RNA splicing.

43. A method according to claim 38 wherein the condition is selected from spinal muscular atrophy, breast cancer, Becker muscular dystrophy and β-thalassaemia.

44. A method of treating a condition characterised by inadequate or defective translation of an RNA species in an individual, the method comprising administering to the individual a nucleic acid molecule having a first domain capable of forming a specific binding pair with a target region of an inadequately or defective translated target RNA species and having a second domain that forms a specific binding pair with an RNA translation factor, wherein said target region of the target RNA species is sufficiently close on the RNA species to a translation initiation site for translation at the site to be enhanced by the action of the translation factor.

45. (canceled)

46. A method according to claim 44 wherein the RNA translation factor is selected from the group consisting of an initiation factor such as eIF4G and eIF3 or a ribosomal component.

47. A method of enhancing polyadenylation at a desired polyadenylation site on a target RNA species, the method comprising:
providing a nucleic acid molecule having a first domain that is capable of forming a first specific binding pair with a target sequence close to the desired polyadenylation site on the target RNA species, and a second domain that is capable of forming a first specific binding pair with an RNA polyadenylation factor, and
contacting the nucleic acid molecule with the target RNA species and with the RNA splicing factor.

48. (canceled)

49. A method according to claim 47 wherein the RNA polyadenylation factor is cleavage and polyadenylation specificity factor (CPSF).

50. (canceled)

51. (canceled)

52. (canceled)

53. A method for the treatment of RNA processing or translation defects caused by mutations in RNA that affect binding of RNA processing or translation factors comprising administering to a patient a nucleic acid molecule having a first domain capable of forming a specific binding pair with a target sequence of a target RNA species and having a second domain capable of forming a second specific binding pair with an RNA processing or translation factor.

54. A method of affecting RNA processing or translation in an in vitro system characterised in the use of a nucleic acid molecule having a first domain capable of forming a specific binding pair with a target sequence of a target RNA species and having a second domain capable of forming a second specific binding pair with an RNA processing or translation factor.

55. A method of designing a nucleic acid molecule that affects RNA processing or translation at an RNA processing or translation site on a target RNA species, the method comprising:
(a) identifying the RNA processing or translation site on the target RNA species, and
(b) designing an oligonucleotide molecule comprising:
(i) a nucleotide sequence that forms a specific binding pair with a target sequence close to the RNA processing or translation site on the target RNA species, and
(ii) a nucleotide sequence motif that forms a specific binding pair with an RNA processing or translation factor which affects processing or translation of the target RNA species at the RNA processing or translation site.

56. A method according to claim 55 further comprising the prior step of selecting a target RNA species.

57. A method of making a nucleic acid molecule that affects RNA processing or translation at an RNA processing or translation site on a target RNA species, the method comprising designing a nucleic acid molecule according to claim 55 and synthesizing the nucleic acid molecule.

58. A method of making a nucleic acid molecule that affects RNA processing or translation at an RNA processing or translation site on a target RNA species, the method comprising designing a nucleic acid molecule according to claim 55 and expressing the nucleic acid molecule from a polynucleotide encoding it.

59. A method according to 55, 57 and 58 wherein the target RNA species is transcribed from a defective or mutated disease gene.

60. A nucleic acid molecule obtainable by the method of claim 57 or 58.


SEQUENCE LISTING (16 sequences)

SEQ ID NO: 1 - DNA, 21 bp, Artificial sequence
  PCR Primer
gggctgctggttgtctaccca

SEQ ID NO: 2 - DNA, 27 bp, Artificial sequence
  PCR Primer
aacttacctgccaaaaatgatgagaca

SEQ ID NO: 3 - DNA, 41 bp, Artificial sequence
  PCR Primer
attcatgttatatggtcgacagactatcaacttaatttctg

SEQ ID NO: 4 - DNA, 35 bp, Artificial sequence
  PCR Primer
gttagcagagtcgacgtgatataaaatggcatatc

SEQ ID NO: 5 - DNA, 26 bp, Artificial sequence
  PCR Primer
cattccttaaatcggagtaagtctgc

SEQ ID NO: 6 - DNA, 27 bp, Artificial sequence
  PCR Primer
gcagacttactcctgatttaaggaatg

SEQ ID NO: 7 - DNA, 42 bp, Artificial sequence
  PCR Primer
aaattaatacgactcactatagggctgctggttgtctaccca

SEQ ID NO: 8 - RNA, 38 nt, Artificial sequence
  modified tailed oligonucleotide
  misc_feature (1)..(23): tail region
  misc_feature (24)..(38): complementary region
agaagaacgaagaacgaagaacagauuuugucuaaaac

SEQ ID NO: 9 - RNA, 38 nt, Artificial sequence
  modified tailed oligonucleotide
  misc_feature (1)..(15): complementary region
  misc_feature (16)..(38): tail region
gauuuugucuaaaacagaagaacgaagaacgaagaaca

SEQ ID NO: 10 - RNA, 38 nt, Artificial sequence
  modified tailed oligonucleotide
  misc_feature (1)..(23): tail region
  misc_feature (24)..(38): complementary region
aggaggacggaggacggaggacagauuuugucuaaaac

SEQ ID NO: 11 - RNA, 38 nt, Artificial sequence
  modified tailed oligonucleotide
  misc_feature (1)..(23): tail region
  misc_feature (24)..(38): complementary region
aucuuuccucuuuccucuuuccagauuuugucuaaaac

SEQ ID NO: 12 - RNA, 38 nt, Artificial sequence
  modified tailed oligonucleotide
  misc_feature (1)..(23): tail region
  misc_feature (24)..(38): complementary region
auagggcaggcuagggcaggccagauuuugucuaaaac

SEQ ID NO: 13 - RNA, 15 nt, Artificial sequence
  modified tailed oligonucleotide
  misc_feature (1)..(15): complementary region
gauuuugucuaaaac

SEQ ID NO: 14 - RNA, 14 nt, Artificial sequence
  modified tailed oligonucleotide
  misc_feature (1)..(14): complementary region
acccugucuuaggu

SEQ ID NO: 15 - RNA, 41 nt, Artificial sequence
  modified tailed oligonucleotide
  misc_feature (1)..(25): tail region
  misc_feature (26)..(41): complementary region
aggaggacggaggacggaggacacgaaaucuguuuuaguuu

SEQ ID NO: 16 - RNA, 23 nt, Artificial sequence
  tail region for modified tailed oligonucleotide
aggaggacggaggacggaggaca